(19) United States
(12) Patent Application Publication
(10) Pub. No.: US 2020/0009211 A1
(43) Pub. Date: Jan. 9, 2020

(54) CURCUMIN-CONTAINING MEDICINAL PREPARATION

(71) Applicants: SAN-EI GEN F.F.I., INC., Toyonaka-shi, Osaka (JP); OSAKA UNIVERSITY, Suita-shi, Osaka (JP)

(72) Inventors: Kazuya NAGANO, Osaka (JP); Kazuo HARADA, Osaka (JP); Kazuma HIGASHISAKA, Osaka (JP); Yasuo TSUTSUMI, Osaka (JP); Tomohiro NAKAO, Osaka (JP)

(21) Appl. No.: 16/489,613

(22) PCT Filed: Mar. 2, 2018
(86) PCT No.: PCT/JP2018/008186

§ 371 Date: Aug. 28, 2019

(30) Foreign Application Priority Data
Mar. 3, 2017 (JP) 2017-041173

(51) Int. Cl. A61K 36/906; A61K 47/14; A61K 47/24; A61K 47/26; A61K 47/32; A61K 47/38

(52) U.S. Cl. CPC A61K 36/906; A61K 47/14; A61K 47/24; A61K 9/0053; A61K 47/32; A61K 47/38; A61K 47/26

(57) ABSTRACT

An object of the present invention is to provide a curcumin-containing solid composition to be used as a composition for treating or preventing diseases and symptoms that benefit from curcumin absorption into cells.

The object is achieved by a solid composition comprising:
(1) curcumin;
(2) hydrophilic polymer; and
(3) at least one nonionic surfactant selected from the group consisting of polyglycerol fatty acid esters, sucrose fatty acid esters, and lecithins.

### TECHNICAL FIELD

The present invention relates to a curcumin-containing preparation and the like.

### BACKGROUND ART

Various studies have confirmed the safety and efficacy of curcumin. It has also been reported that curcumins are considered to have various physiological effects, such as suppression of cholesterol elevation, suppression of blood pressure elevation, suppression of blood glucose elevation, anti-allergy, suppression of body fat, and the like.

To expect such physiological effects, the ingestion of large amounts of curcumin is necessary.

Curcumins are components contained in, for example, edible plants. Although curcumins can be ingested, for example, in usual meals, ingesting curcumins in the form of curcumin-containing tablets or like solid preparations is convenient and efficient.

However, most curcumins are poorly soluble in water. Therefore, even if a curcumin-containing solid preparation is ingested, curcumins are dissolved and absorbed into the body fluid at slow rates.

To solve this problem, for example, Patent Document 1 suggests a preparation for oral administration comprising a curcuminoid and an essential oil of turmeric.

Further, several strategies have been attempted to improve the bioavailability of curcumin, such as regulation of curcumin administration route and media, blockade of metabolic pathways by co-administration with other drugs, modification of the bond and structure of curcumin, and the like.

Despite these attempts, the use of curcumin for human health is actually limited. This strongly suggests the need for further improvements in the bioavailability of curcumin.

Therefore, the development of a new technique has been desired, in view of the efficient ingestion of curcumins.

### CITATION LIST

### Patent Documents

Patent Document 1: JP2012-188450A

### SUMMARY OF INVENTION

### Technical Problem

An object of the present invention is to provide a preparation for treating or preventing diseases or symptoms that benefit from curcumin absorption into cells.

### Solution to Problem

The inventors of the present invention carried out extensive research to solve the problems, and found that efficient curcumin ingestion (incorporation into cells etc.) becomes possible by a preparation comprising a solid composition comprising:
(1) curcumin;
(2) hydrophilic polymer; and
(3) at least one nonionic surfactant selected from the group consisting of polyglycerol fatty acid esters, sucrose fatty acid esters, and lecithins.

Based on this finding, the inventors also found that the problems can be solved by this preparation, and completed the present invention.

The present invention encompasses the following aspects.

### Item 1

A preparation for treating or preventing a disease or symptom that benefits from curcumin absorption into cells, the preparation comprising a solid composition comprising:
(1) curcumin;
(2) hydrophilic polymer; and
(3) at least one nonionic surfactant selected from the group consisting of polyglycerol fatty acid esters, sucrose fatty acid esters, and lecithins.

### Item 2

The preparation according to item 1, wherein the disease or symptom is at least one member selected from the group consisting of:
(1) diseases or symptoms that benefit from modulation of one or more transcription factors selected from the group consisting of NF-κB, AP-1, STAT, Wnt/β-catenin, Notch-1, EGR-1, CREB-BP, WT-1, HIF, ERE, Nrf-2, PPAR-α, and PPAR-γ;
(2) diseases or symptoms that benefit from modulation of one or more cytokines selected from the group consisting of TNF-α, IL-1β, IL-2, IL-5, IL-6, IL-8, IL-12, IL-18, MCP-1, MIP-1α, and MaIP;
(3) diseases or symptoms that benefit from modulation of one or more receptors selected from the group consisting of IR, ER-α, H2R, HER-2, LDLR, ITR, FasR, EPCR, AR, EGFR, IL-8R, CXCR4, AHR, and DR-5;
(4) diseases or symptoms that benefit from modulation of one or more enzymes selected from the group consisting of Desaturase, GCL, AATF-1, ATFase, Telomerase, MMP, ATPase, GICL, COX-2, iNOS, NQO-1, 5-LOX, TMMP-3, DNA pol, Src-2, FPT, PhP D, GST, ODC, and ACOX-1;
(5) diseases or symptoms that benefit from modulation of one or more growth factors selected from the group consisting of HGF, CTGF, FGF, NGF, PDGF, TGF-β1, EGF, VEGF, and TF;
(6) diseases or symptoms that benefit from modulation of one or more kinases selected from the group consisting of FAK, AAPK, P60c-tk, EGFR-K, Ca²⁺PK, PTK, MAPK, IL-1R AK, PKB, PKA, PAK, JAK, ERK, PhK, and JNK;
(7) diseases or symptoms that benefit from modulation of one or more members selected from the group consisting of uPA, Bcl-2, Bcl-xL, VCAM-1, ICAM-1, ELAM-1, IAP-1, Hsp-70, Cyclin D1, MDRP, p53, and DEF-40;
(8) diseases or symptoms that benefit from inhibition of aggregation of amyloid β;
(9) diseases or symptoms that benefit from suppression of aggregation of α-synuclein;
(10) diseases or symptoms that benefit from a decrease of one or more members selected from the group consisting of ALT, AST, and γ-GTP;
(11) diseases or symptoms that benefit from inhibition of HAT activity of p300;
(12) diseases or symptoms that benefit from antioxidant effects; and
(13) diseases or symptoms that benefit from suppression of an increase in acetaldehyde concentration due to alcohol ingestion.

### Item 3

The preparation according to item 1, wherein the treatment or prevention of a disease or symptom is at least one member selected from the group consisting of:
(1) treatment or prevention of cancers or tumors;
(2) treatment or prevention of diabetes;
(3) treatment or prevention of hyperglycemia;
(4) treatment or prevention of periodontitis;
(5) treatment or prevention of Alzheimer's disease or mild cognitive impairment;
(6) treatment or prevention of Parkinson's disease;
(7) treatment or prevention of nerve disorder;
(8) treatment or prevention of inflammation;
(9) treatment or prevention of amyloidosis;
(10) protection of liver function;
(11) treatment or prevention of heart failure;
(12) treatment or prevention of myocardial infarction;
(13) treatment or prevention of muscle fatigue;
(14) protection of kidney function;
(15) treatment or prevention of osteoporosis;
(16) treatment or prevention of depression;
(17) treatment or prevention of multiple sclerosis;
(18) treatment or prevention of ischemia; and
(19) treatment or prevention of hangover symptoms after alcohol ingestion.

### Item 4

The preparation according to item 1, wherein the treatment or prevention of a disease or symptom is at least one member selected from the group consisting of suppression of cholesterol elevation, suppression of triglyceride elevation, suppression of chylomicron elevation, suppression of blood pressure elevation, suppression of blood glucose elevation, anti-allergy, and suppression of body fat.

### Item 5

The preparation according to any one of items 1 to 4, wherein the hydrophilic polymer is at least one member selected from the group consisting of polyvinylpyrrolidone, hydroxypropyl cellulose, and hydroxypropyl methylcellulose.

### Item 6

The preparation according to any one of items 1 to 5, wherein the nonionic surfactant is a polyglycerol fatty acid ester.

### Item 7

The preparation according to any one of items 1 to 6, wherein the preparation is a preparation for oral administration, a preparation for gastrointestinal administration, a preparation for transdermal administration, or a preparation for pulmonary administration.

### Item 8

The preparation according to any one of items 1 to 7, wherein the preparation is a pharmaceutical product, quasi-drug, health food, food with function claims, dietary supplement, food with nutrient function claims, nutritional supplement, food for special dietary use, or a food for specified health use.

### Item A1

A method for treating or preventing a disease or symptom that benefits from curcumin absorption into cells, the method comprising administering a solid composition comprising:
(1) curcumin;
(2) hydrophilic polymer; and
(3) at least one nonionic surfactant selected from the group consisting of polyglycerol fatty acid esters, sucrose fatty acid esters, and lecithins,

to a subject in need thereof.

### Item A2

The method according to item Al, wherein the disease or symptom is at least one member selected from the group consisting of:
(1) diseases or symptoms that benefit from modulation of one or more transcription factors selected from the group consisting of NF-κB, AP-1, STAT, Wnt/β-catenin, Notch-1, EGR-1, CREB-BP, WT-1, HIF, ERE, Nrf-2, PPAR-α, and PPAR-γ;
(2) diseases or symptoms that benefit from modulation of one or more cytokines selected from the group consisting of TNF-α, IL-1β, IL-2, IL-5, IL-6, IL-8, IL-12, IL-18, MCP-1, MIP-1α, and MaIP;
(3) diseases or symptoms that benefit from modulation of one or more receptors selected from the group consisting of IR, ER-α, H2R, HER-2, LDLR, ITR, FasR, EPCR, AR, EGFR, IL-8R, CXCR4, AHR, and DR-5;
(4) diseases or symptoms that benefit from modulation of one or more enzymes selected from the group consisting of Desaturase, GCL, AATF-1, ATFase, Telomerase, MMP, ATPase, GICL, COX-2, iNOS, NQO-1, 5-LOX, TMMP-3, DNA pol, Src-2, FPT, PhP D, GST, ODC, and ACOX-1;
(5) diseases or symptoms that benefit from modulation of one or more growth factors selected from the group consisting of HGF, CTGF, FGF, NGF, PDGF, TGF-β1, EGF, VEGF, and TF;
(6) diseases or symptoms that benefit from modulation of one or more kinases selected from the group consisting of FAK, AAPK, P60c-tk, EGFR-K, Ca²⁺PK, PTK, MAPK, IL-1R AK, PKB, PKA, PAK, JAK, ERK, PhK, and JNK;
(7) diseases or symptoms that benefit from modulation of one or more members selected from the group consisting of uPA, Bcl-2, Bcl-xL, VCAM-1, ICAM-1, ELAM-1, IAP-1, Hsp-70, Cyclin D1, MDRP, p53, and DEF-40;
(8) diseases or symptoms that benefit from inhibition of aggregation of amyloid β;
(9) diseases or symptoms that benefit from suppression of aggregation of α-synuclein;
(10) diseases or symptoms that benefit from a decrease of one or more members selected from the group consisting of ALT, AST, and γ-GTP;
(11) diseases or symptoms that benefit from inhibition of HAT activity of p300;
(12) diseases or symptoms that benefit from antioxidant effects; and
(13) diseases or symptoms that benefit from suppression of an increase in acetaldehyde concentration due to alcohol ingestion.

### Item A3

The method according to item A1, wherein the treatment or prevention of a disease or symptom is at least one member selected from the group consisting of:
(1) treatment or prevention of cancers or tumors;
(2) treatment or prevention of diabetes;
(3) treatment or prevention of hyperglycemia;
(4) treatment or prevention of periodontitis;
(5) treatment or prevention of Alzheimer's disease or mild cognitive impairment;
(6) treatment or prevention of Parkinson's disease;
(7) treatment or prevention of nerve disorder;
(8) treatment or prevention of inflammation;
(9) treatment or prevention of amyloidosis;
(10) protection of liver function;
(11) treatment or prevention of heart failure;
(12) treatment or prevention of myocardial infarction;
(13) treatment or prevention of muscle fatigue;
(14) protection of kidney function;
(15) treatment or prevention of osteoporosis;
(16) treatment or prevention of depression;
(17) treatment or prevention of multiple sclerosis;
(18) treatment or prevention of ischemia; and
(19) treatment or prevention of hangover symptoms after alcohol ingestion.

### Item A4

The method according to item A1, wherein the treatment or prevention of a disease or symptom is at least one member selected from the group consisting of suppression of cholesterol elevation, suppression of triglyceride elevation, suppression of chylomicron elevation, suppression of blood pressure elevation, suppression of blood glucose elevation, anti-allergy, and suppression of body fat.

### Item A5

The method according to any one of items A1 to A4, wherein the hydrophilic polymer is at least one member selected from the group consisting of polyvinylpyrrolidone, hydroxypropyl cellulose, and hydroxypropyl methylcellulose.

### Item A6

The method according to any one of items A1 to A5, wherein the nonionic surfactant is a polyglycerol fatty acid ester.

### Item A7

The method according to any one of items A1 to A6, wherein the administration is oral administration, administration through gastrointestinal tract, transdermal administration, or pulmonary administration.

### Item A8

The method according to any one of Items A1 to A7, wherein a pharmaceutical product, quasi-drug, health food, food with function claims, dietary supplement, food with nutrient function claims, nutritional supplement, food for special dietary use, or a food for specified health use is administered in the method.

### Item B1

A solid composition for treating or preventing a disease or symptom that benefits from curcumin absorption into cells, the solid composition comprising:
(1) curcumin;
(2) hydrophilic polymer; and
(3) at least one nonionic surfactant selected from the group consisting of polyglycerol fatty acid esters, sucrose fatty acid esters, and lecithins.

### Item B2

The solid composition according to item B1, wherein the disease or symptom is at least one member selected from the group consisting of:
(1) diseases or symptoms that benefit from modulation of one or more transcription factors selected from the group consisting of NF-κB, AP-1, STAT, Wnt/β-catenin, Notch-1, EGR-1, CREB-BP, WT-1, HIF, ERE, Nrf-2, PPAR-α, and PPAR-γ;
(2) diseases or symptoms that benefit from modulation of one or more cytokines selected from the group consisting of TNF-α, IL-1β, IL-2, IL-5, IL-6, IL-8, IL-12, IL-18, MCP-1, MIP-1α, and MaIP;
(3) diseases or symptoms that benefit from modulation of one or more receptors selected from the group consisting of IR, ER-α, H2R, HER-2, LDLR, ITR, FasR, EPCR, AR, EGFR, IL-8R, CXCR4, AHR, and DR-5;
(4) diseases or symptoms that benefit from modulation of one or more enzymes selected from the group consisting of Desaturase, GCL, AATF-1, ATFase, Telomerase, MMP, ATPase, GICL, COX-2, iNOS, NQO-1, 5-LOX, TMMP-3, DNA pol, Src-2, FPT, PhP D, GST, ODC, and ACOX-1;
(5) diseases or symptoms that benefit from modulation of one or more growth factors selected from the group consisting of HGF, CTGF, FGF, NGF, PDGF, TGF-β1, EGF, VEGF, and TF;
(6) diseases or symptoms that benefit from modulation of one or more kinases selected from the group consisting of FAK, AAPK, P60c-tk, EGFR-K, Ca²⁺PK, PTK, MAPK, IL-1R AK, PKB, PKA, PAK, JAK, ERK, PhK, and JNK;
(7) diseases or symptoms that benefit from modulation of one or more members selected from the group consisting of uPA, Bcl-2, Bcl-xL, VCAM-1, ICAM-1, ELAM-1, IAP-1, Hsp-70, Cyclin D1, MDRP, p53, and DEF-40;
(8) diseases or symptoms that benefit from inhibition of aggregation of amyloid β;
(9) diseases or symptoms that benefit from suppression of aggregation of α-synuclein;
(10) diseases or symptoms that benefit from a decrease of one or more members selected from the group consisting of ALT, AST, and γ-GTP;
(11) diseases or symptoms that benefit from inhibition of HAT activity of p300;
(12) diseases or symptoms that benefit from antioxidant effects; and
(13) diseases or symptoms that benefit from suppression of an increase in acetaldehyde concentration due to alcohol ingestion.

### Item B3

The solid composition according to item B1, wherein the treatment or prevention of a disease or symptom is at least one member selected from the group consisting of:
(1) treatment or prevention of cancers or tumors;
(2) treatment or prevention of diabetes;
(3) treatment or prevention of hyperglycemia;
(4) treatment or prevention of periodontitis;
(5) treatment or prevention of Alzheimer's disease or mild cognitive impairment;
(6) treatment or prevention of Parkinson's disease;
(7) treatment or prevention of nerve disorder;
(8) treatment or prevention of inflammation;
(9) treatment or prevention of amyloidosis;
(10) protection of liver function;
(11) treatment or prevention of heart failure;
(12) treatment or prevention of myocardial infarction;
(13) treatment or prevention of muscle fatigue;
(14) protection of kidney function;
(15) treatment or prevention of osteoporosis;
(16) treatment or prevention of depression;
(17) treatment or prevention of multiple sclerosis;
(18) treatment or prevention of ischemia; and
(19) treatment or prevention of hangover symptoms after alcohol ingestion.

### Item B4

The solid composition according to item B1, wherein the treatment or prevention of a disease or symptom is at least one member selected from the group consisting of suppression of cholesterol elevation, suppression of triglyceride elevation, suppression of chylomicron elevation, suppression of blood pressure elevation, suppression of blood glucose elevation, anti-allergy, and suppression of body fat.

### Item B5

The solid composition according to any one of items B1 to B4, wherein the hydrophilic polymer is at least one member selected from the group consisting of polyvinylpyrrolidone, hydroxypropyl cellulose, and hydroxypropyl methylcellulose.

### Item B6

The solid composition according to any one of items B1 to B5, wherein the nonionic surfactant is a polyglycerol fatty acid ester.

### Item B7

The solid composition according to any one of items B1 to B6, wherein the solid composition is a solid composition for oral administration, a solid composition for gastrointestinal administration, a solid composition for transdermal administration, or a solid composition for pulmonary administration.

### Item B8

The solid composition according to any one of items B1 to B7, wherein the solid composition is a pharmaceutical product, quasi-drug, health food, food with function claims, dietary supplement, food with nutrient function claims, nutritional supplement, food for special dietary use, or a food for specified health use.

### Item C1

A composition for the manufacture of a preparation for treating or preventing a disease or symptom that benefits from curcumin absorption into cells, the composition comprising a solid composition comprising:
(1) curcumin;
(2) hydrophilic polymer; and
(3) at least one nonionic surfactant selected from the group consisting of polyglycerol fatty acid esters, sucrose fatty acid esters, and lecithins.

### Item C2

The composition according to item Cl, wherein the disease or symptom is at least one member selected from the group consisting of:
(1) diseases or symptoms that benefit from modulation of one or more transcription factors selected from the group consisting of NF-κB, AP-1, STAT, Wnt/β-catenin, Notch-1, EGR-1, CREB-BP, WT-1, HIF, ERE, Nrf-2, PPAR-α, and PPAR-γ;
(2) diseases or symptoms that benefit from modulation of one or more cytokines selected from the group consisting of TNF-α, IL-1β, IL-2, IL-5, IL-6, IL-8, IL-12, IL-18, MCP-1, MIP-1α, and MaIP;
(3) diseases or symptoms that benefit from modulation of one or more receptors selected from the group consisting of IR, ER-α, H2R, HER-2, LDLR, ITR, FasR, EPCR, AR, EGFR, IL-8R, CXCR4, AHR, and DR-5;
(4) diseases or symptoms that benefit from modulation of one or more enzymes selected from the group consisting of Desaturase, GCL, AATF-1, ATFase, Telomerase, MMP, ATPase, GICL, COX-2, iNOS, NQO-1, 5-LOX, TMMP-3, DNA pol, Src-2, FPT, PhP D, GST, ODC, and ACOX-1;
(5) diseases or symptoms that benefit from modulation of one or more growth factors selected from the group consisting of HGF, CTGF, FGF, NGF, PDGF, TGF-β1, EGF, VEGF, and TF;
(6) diseases or symptoms that benefit from modulation of one or more kinases selected from the group consisting of FAK, AAPK, P60c-tk, EGFR-K, Ca²⁺PK, PTK, MAPK, IL-1R AK, PKB, PKA, PAK, JAK, ERK, PhK, and JNK;
(7) diseases or symptoms that benefit from modulation of one or more members selected from the group consisting of uPA, Bcl-2, Bcl-xL, VCAM-1, ICAM-1, ELAM-1, IAP-1, Hsp-70, Cyclin D1, MDRP, p53, and DEF-40;
(8) diseases or symptoms that benefit from inhibition of aggregation of amyloid β;
(9) diseases or symptoms that benefit from suppression of aggregation of α-synuclein;
(10) diseases or symptoms that benefit from a decrease of one or more members selected from the group consisting of ALT, AST, and γ-GTP;
(11) diseases or symptoms that benefit from inhibition of HAT activity of p300;
(12) diseases or symptoms that benefit from antioxidant effects; and
(13) diseases or symptoms that benefit from suppression of an increase in acetaldehyde concentration due to alcohol ingestion.

### Item C3

The composition according to item C1, wherein the treatment or prevention of a disease or symptom is at least one member selected from the group consisting of:
(1) treatment or prevention of cancers or tumors;
(2) treatment or prevention of diabetes;
(3) treatment or prevention of hyperglycemia;
(4) treatment or prevention of periodontitis;
(5) treatment or prevention of Alzheimer's disease or mild cognitive impairment;
(6) treatment or prevention of Parkinson's disease;
(7) treatment or prevention of nerve disorder;
(8) treatment or prevention of inflammation;
(9) treatment or prevention of amyloidosis;
(10) protection of liver function;
(11) treatment or prevention of heart failure;
(12) treatment or prevention of myocardial infarction;
(13) treatment or prevention of muscle fatigue;
(14) protection of kidney function;
(15) treatment or prevention of osteoporosis;
(16) treatment or prevention of depression;
(17) treatment or prevention of multiple sclerosis;
(18) treatment or prevention of ischemia; and
(19) treatment or prevention of hangover symptoms after alcohol ingestion.

### Item C4

The composition according to item C1, wherein the treatment or prevention of a disease or symptom is at least one member selected from the group consisting of suppression of cholesterol elevation, suppression of triglyceride elevation, suppression of chylomicron elevation, suppression of blood pressure elevation, suppression of blood glucose elevation, anti-allergy, and suppression of body fat.

### Item C5

The composition according to any one of items C1 to C4, wherein the hydrophilic polymer is at least one member selected from the group consisting of polyvinylpyrrolidone, hydroxypropyl cellulose, and hydroxypropyl methylcellulose.

### Item C6

The composition according to any one of items C1 to C5, wherein the nonionic surfactant is a polyglycerol fatty acid ester.

### Item C7

The composition according to any one of items C1 to C6, wherein the preparation is a preparation for oral administration, a preparation for gastrointestinal administration, a preparation for transdermal administration, or a preparation for pulmonary administration.

### Item C8

The composition according to any one of items C1 to C7, wherein the preparation is a pharmaceutical product, quasi-drug, health food, food with function claims, dietary supplement, food with nutrient function claims, nutritional supplement, food for special dietary use, or a food for specified health use.

### Advantageous Effects of Invention

When orally administered or ingested, the preparation of the present invention allows curcumin to dissolve at a high rate into the body fluid (preferably the intestinal fluid) for a prolonged period of time, to thereby enable efficient ingestion of the curcumin.

The preparation of the present invention more easily enables curcumin contained therein to be incorporated into cells.

More specifically, the present invention makes it possible to provide a curcumin-containing preparation that enables efficient ingestion of curcumin.

Further, such a curcumin-containing preparation also enables provision of an excellent composition for treating or preventing diseases or symptoms that benefit from curcumin absorption into cells.

### BRIEF DESCRIPTION OF DRAWINGS

FIG. 1 is a graph showing the changes over time of the dissolution of curcumin into artificial intestinal fluid from the curcumin-containing preparation of Example 1, in comparison with a solid composition not containing a nonionic surfactant (Comparative Example 1), a solubilized preparation (Comparative Example 2), and a solid composition prepared without heating (Comparative Example 3).

FIG. 2 is a graph showing the changes over time of the dissolution of curcumin into artificial intestinal fluid from the preparation of Example 1, in comparison with various non-ionic surfactants (Comparative Examples 4 to 7) other than the non-ionic surfactant used in the present invention.

FIG. 3 is a graph showing the changes over time of the dissolution of curcumin into artificial intestinal fluid from curcumin-containing preparations (Examples 1 to 3) comprising various types of polyglycerol fatty acid esters.

FIG. 4 is a graph showing the changes over time of the dissolution of curcumin into artificial intestinal fluid from curcumin-containing preparations comprising a polyglycerol fatty acid ester in various amounts (Examples 1, 4, 5, and Comparative Example 1).

FIG. 5 is a graph showing the changes over time of the dissolution of curcumin into artificial intestinal fluid from curcumin-containing preparations (Examples 1 and 6 to 9) comprising various nonionic surfactants.

FIG. 6 is a graph showing the changes over time of the blood curcumin concentration in rats to which the curcumin-containing preparation of Example 1 was administered, in comparison with that in rats to which a curcumin bulk powder was administered as a comparative example.

FIG. 7-1 is a graph showing the cytotoxicity test results of B16F10 (skin cancer cells) (Test Example 8-1).

FIG. 7-2 is a graph showing the cytotoxicity test results of HaCaT (human epidermal keratinocytes) (Test Example 8-1).

FIG. 7-3 is a graph showing the B16F10/HaCaT ratio in the cytotoxicity test (Test Example 8-1).

FIG. 8 is a graph showing the cytotoxicity test results of MDA-MB-436 (breast cancer cells) (Test Example 8-2).

FIG. 9 is a graph showing the cytotoxicity test results of EL-4 (lymphoma cells) (Test Example 8-3).

FIG. 10 is a graph showing the cytotoxicity test results of A-549 (lung cancer cells) (Test Example 8-4).

FIG. 11 is a graph showing the cytotoxicity test results of B16F10 (skin cancer cells) (Test Example 8-5).

FIG. 12 is a graph showing the insulin secretion test results of MIN6 cells (mouse pancreatic β cells) (Test Example 9).

FIG. 13-1 is graphs showing the results of organ weight measurement in acute toxicity tests (Test Example 10).

FIG. 13-2 is graphs showing the results of biochemical testing in acute toxicity tests (Test Example 10).

FIG. 13-3 is graphs showing the results of blood cell testing in acute toxicity tests (Test Example 10).

FIG. 14 is graphs showing the test (TCHO, CM, LDL) results (A) of administering curcumin preparations to HFD-challenged mice (Test Example 11).

FIG. 15 is a graph showing the evaluation results of curcumin biodistribution after administering a curcumin preparation (Test Example 12).

FIG. 16 is a graph showing the evaluation results of curcumin tissue distribution by long-term ingestion of a curcumin preparation (after 3 months of administration by ingestion) (Test Example 13).

FIG. 17 is a graph showing the evaluation results of curcumin tissue distribution by long-term ingestion of curcumin preparations (2 hours after ceasing the administration by ingestion) (Test Example 13).

FIG. 18 is a graph showing the test (TG) results (B) of administering curcumin preparations to HFD-challenged mice (Test Example 14).

FIG. 19 is a graph showing the results of mouse liver weight measurement after administering curcumin preparations (Test Example 15).

FIG. 20 is a graph showing the mRNA expression level of ACOX1 after administering curcumin preparations (Test Example 16).

FIG. 21 is a graph showing the urinary curcumin concentration after administering curcumin preparations (Test Example 17).

FIG. 22 is a graph showing the urinary curcumin (including a glucuronide conjugate) concentration after administering curcumin preparations (Test Example 17).

### DESCRIPTION OF EMBODIMENTS

### Terms

The symbols and abbreviations used in this specification can be assumed to have their ordinary meanings used in the technical field to which the present invention pertains, as understood from the context of the specification, unless otherwise specified.

In the specification, the terms “containing” and “comprising” are used to include meanings of the phrase “consisting essentially of” and the phrase “consisting of.”

The step, treatment, or operation disclosed in the specification can be performed at room temperature, unless otherwise specified. In this specification, room temperature refers to a temperature in the range of 10 to 40° C.

### 1. Preparation

The preparation of the present invention comprises a solid composition containing:
(1) curcumin;
(2) hydrophilic polymer; and
(3) at least one nonionic surfactant selected from the group consisting of polyglycerol fatty acid esters, sucrose fatty acid esters, and lecithins.

The preparation of the present invention encompasses a preparation essentially consisting of the solid composition, and a preparation consisting of the solid composition.

### (1) Curcumin

In general, curcumins are crystalline, and are thus poorly soluble in water or insoluble in water.

“Poorly water-soluble” as used herein specifically means that the solubility in pure water is 0.1 mass % or less at 25° C., or that the octanol/water partition coefficient (logP) falls within the range of −1.0 to 4.0. The logP value can be determined by high-performance liquid chromatography according to JIS Z 7260-117 (2006).

The logP value is defined by the following formula:

logP=log(Coc/Cwa)

Coc: concentration of the test substance in the 1-octanol layer

Cwa: concentration of the test substance in the aqueous layer

The “curcumin” used in the present invention may have a solubility of 0.2 mg/100 mL or less with respect to the second dissolution medium of the Japanese Pharmacopoeia, 16th edition, which is determined in accordance with the method prescribed in the Japanese Pharmacopoeia dissolution test.

The curcumin contained in the solid composition may be, for example, an extract derived from a natural product (e.g., turmeric extract), or a synthetic product.

The curcumin contained in the solid composition may have a keto form, an enol form, or a mixture thereof.

The curcumin content in the solid composition is preferably within the range of 1 to 60 mass %, more preferably within the range of 5 to 50 mass %, further preferably 7 to 40 mass %, and even more preferably within the range of 10 to 35 mass %.

Although the solid composition may contain crystalline curcumin, the amount or the proportion of the crystalline curcumin relative to the entire solid composition or total curcumins is preferably small.

The amorphous state of the curcumin contained in the solid composition can be confirmed by powder X-ray diffraction, differential scanning calorimetry, or like methods. Further, the amount of the amorphous curcumin can be calculated from the peak areas of differential scanning calorimetry.

It is particularly preferable that the solid composition is substantially or entirely free of crystalline curcumin. In other words, the curcumin contained in the solid composition of the present invention is preferably substantially amorphous.

The amount of total curcumins in the solid composition (the “total curcumins” include curcumins and crystalline curcumins) is preferably within the range of 1 to 60 mass %, more preferably within the range of 5 to 50 mass %, further preferably 7 to 40 mass %, and even more preferably within the range of 10 to 35 mass %.

### (2) Hydrophilic Polymer

The hydrophilic polymer used in the present invention is not necessarily hydrophilic or water-soluble under every condition. The hydrophilic polymer is preferably hydrophilic or water-soluble at least at the pH in the intestinal tract.

The hydrophilic polymer used in the present invention is preferably a solid at room temperature.

The hydrophilic polymer used in the present invention preferably has a glass transition temperature (Tg) of preferably about 50° C. or more, more preferably about 80° C. to about 180° C. The determination of the glass transition temperature (Tg) can be performed according to JIS K 7121: 2012.

The solid composition may contain one hydrophilic polymer, or two or more hydrophilic polymers.

Examples of the hydrophilic polymer used in the present specification include the followings.
(1) homopolymers of N-vinyllactam (preferably N-vinylpyrrolidone) (e.g., polyvinylpyrrolidones (i.e., PVP or povidone) (e.g., Kollidon™ 12PF, Kollidon™ 17PF, Kollidon™ 25, Kollidon™ 30, Kollidon™ 90F, or equivalents thereof)), and copolymers thereof (e.g., a copolymer of N-vinylpyrrolidone and vinyl acetate monomers (i.e., copovidone), or a copolymer of N-vinylpyrrolidone and vinyl propionate monomers);
(2) cellulose esters and cellulose ethers, in particular, methyl cellulose, ethyl cellulose, hydroxyalkyl cellulose (e.g., hydroxypropyl cellulose (i.e., HPC)), hydroxyalkyl alkyl cellulose (e.g., hydroxypropyl methylcellulose (namely, HPMC)), or hypromellose (e.g., Methocel™ E3, Methocel™ E5, Methocel™ E6, Methocel™ E15, or equivalents thereof; and Methocel™ K3, or equivalents thereof), cellulose phthalate, and cellulose succinate (e.g., cellulose acetate phthalate, hydroxypropyl methylcellulose phthalate, hydroxypropyl methylcellulose succinate, and hydroxypropyl methylcellulose acetate succinate (i.e., HPMC-AS));
(3) high-molecular-weight polyalkylene oxides (e.g., polyethylene oxide, polypropylene oxide, and ethylene oxide, and copolymers of propylene oxide (e.g., poloxamers));
(4) polyacrylate and polymethacrylate (e.g., methacrylic acid/ethyl acrylate copolymer, methacrylic acid/methyl methacrylate copolymer, butyl methacrylate/2-dimethylaminoethyl methacrylate copolymer, poly(hydroxyalkyl acrylate), and poly(hydroxyalkyl methacrylate));
(5) polyacrylamides;
(6) vinyl acetate copolymers and copolymers of polyvinyl alcohol; oligosaccharide and polysaccharides (e.g., carrageenan, galactomannan, and xanthan gum);
   and mixtures of two or more of the above compounds.

In one preferable embodiment of the present invention, the solid composition may contain, as the hydrophilic polymer, at least one member selected from the group consisting of polyvinylpyrrolidone, hydroxypropyl cellulose, and hydroxypropyl methylcellulose; and may further contain other hydrophilic polymers.

In one particularly preferable embodiment of the present invention, the solid composition may contain at least a polyvinylpyrrolidone as the hydrophilic polymer, and may further contain other hydrophilic polymers.

In another preferable embodiment of the present invention, the hydrophilic polymer is at least one member selected from the group consisting of polyvinylpyrrolidone, hydroxypropyl cellulose, and hydroxypropyl methylcellulose.

In another particularly preferable embodiment, the hydrophilic polymer is-polyvinylpyrrolidone.

The hydrophilic polymer content of the solid composition is preferably within the range of 5 to 90 mass %, more preferably within the range of 20 to 90 mass %, and even more preferably within the range of 40 to 90 mass %.

### (3) Nonionic Surfactant

The nonionic surfactant contained in the solid composition is a nonionic surfactant that is at least one member selected from the group consisting of polyglycerol fatty acid esters, sucrose fatty acid esters, and lecithins.

Examples of polyglycerol fatty acid esters used in the present invention include esters of (a) polyglycerols having an average degree of polymerization of 2 or more (preferably 3 to 15, more preferably 3 to 10), and (b) fatty acids having 8 to 18 carbon atoms (e.g., caprylic acid, capric acid, lauric acid, myristic acid, palmitic acid, stearic acid, oleic acid, and linoleic acid).

Specific examples of polyglycerol fatty acid esters used in the present invention include diglycerol monolaurate, diglycerol monostearate, diglycerol monooleate, decaglycerol monolaurate, decaglycerol monostearate, and decaglycerol monooleate.

In the present invention, the polyglycerol fatty acid esters can be used singly, or in a combination of two or more.

The HLB value of sucrose fatty acid esters used in the present invention is preferably 5 or more, more preferably 7 or more, further preferably 10 or more, and further more preferably 12 or more.

The fatty acid of the sucrose fatty acid ester used in the present invention preferably has at least 12 carbon atoms, and more preferably 12 to 20 carbon atoms.

Specific examples of sucrose fatty acid esters preferably used in the present invention include sucrose laurate, sucrose myristate, sucrose palmitate, sucrose stearate, sucrose oleate, sucrose behenate, and sucrose erucate.

In the present invention, the sucrose fatty acid esters can be used singly, or in a combination of two or more.

The lecithin used in the present invention is an adduct of a phosphoric acid derivative of di-fatty acid ester of glycerol (diglyceride). Lecithin is widely distributed in plant and animal bodies.

Examples of the lecithin used in the present invention include egg yolk lecithin contained in egg yolk, soybean lecithin contained in soybeans, and sunflower lecithin contained in sunflowers.

Examples of the lecithin used in the present invention include fractionated lecithin obtained by extracting an active ingredient from a lecithin described above, enzymatically modified lecithin obtained by treating lecithin with an enzyme, and enzymatically decomposed lecithin.

Specific examples of the lecithin used in the present invention include lecithin, enzymatically decomposed lecithin (phosphatidic acid), lysolecithin, soybean lecithin (soybean phospholipid), and egg yolk lecithin.

Lecithins that can be used in the present invention are commercially available. For example, SLP-White (trade name, produced by Tsuji Oil Mill Co., Ltd.) can be used.

In the present invention, lecithins can be used singly, or in a combination of two or more.

Particularly suitable examples of the nonionic surfactant contained in the solid composition include polyglycerol fatty acid esters.

The solid composition may contain one or more nonionic surfactants.

In a preferred embodiment of the present invention, the nonionic surfactant is a polyglycerol fatty acid ester.

The nonionic surfactant content in the solid composition is preferably within the range of 5 to 90 mass %, more preferably within the range of 5 to 60 mass %, and further preferably within the range of 10 to 40 mass %.

### (4) Other Components

If necessary, the solid composition may contain components other than those mentioned above, as long as the effects of the present invention are not significantly impaired.

Examples of such components include excipients, fillers, extenders, binders, disintegrators, surfactants, seasonings, flavoring agents, and lubricants.

As long as the effect of the present invention is not significantly impaired, the types and amounts of such components may be suitably selected and designed based on common general technical knowledge.

### 2. Use and Form of Preparation

The preparation of the present invention may be used as a pharmaceutical product, quasi-drug, health food, food with function claims, dietary supplement (supplement), food with nutrient function claims, nutritional supplement, food for special dietary use, a food for specified health use, or the like.

The preparation of the present invention may be a preparation for oral administration, a preparation to be applied to the oral cavity, a preparation to be applied to bronchus and lung, a preparation to be applied to eyes, a preparation to be applied to ears, a preparation to be applied to nose, a preparation to be applied to rectum, a preparation to be applied to vagina, or a preparation to be applied to skin.

Further, the preparation of the present invention may preferably be a preparation for oral administration, a preparation for gastrointestinal administration, a preparation for transdermal administration, or a preparation for pulmonary administration; and more preferably a preparation for oral administration.

Examples of suitable forms of the preparation include tablets (e.g., orally disintegrating tablets, chewable tablets, effervescent tablets, dispersible tablets, soluble tablets), capsules, granules (e.g., effervescent granules), powdered drug, liquids and solutions for oral administration (e.g., elixirs, suspensions, emulsions, limonades), syrups (e.g., preparations for syrups), jellies for oral administration, tablets for oro-mucosal application (e.g., troches, sublingual tablets, buccal tablets, mucoadhesive tablets, medicated chewing gum), sprays for oro-mucosal application, semi-solid preparations for oro-mucosal application, preparations for gargle, dialysis agents (e.g., peritoneal dialysis agents, hemodialysis agents), inhalant solutions (e.g., dry powder inhalers, inhalation solutions, inhalation aerosols), suppositories, semi-solid preparations for rectal application, enemas for rectal application, ophthalmic ointments, ear preparations, nasal preparations (e.g., nasal dry powder inhalers, nasal liquids and solutions), vaginal tablets, vaginal suppositories, solid preparations for cutaneous application (e.g., powders for cutaneous application), liquids and solutions for cutaneous application (e.g., liniments, lotions), and sprays (e.g., aerosols for cutaneous application, pump sprays).

When the preparation is a cosmetic or a similar product, examples of suitable forms thereof include aqueous lotions (e.g., lotions), emulsions, and creams.

Further, examples of the preparations of the present invention include dental care products (e.g., toothpaste) and oral care products (e.g., mouthwash).

These preparations may be produced based on common technical knowledge related to the manufacture of preparations containing a solid composition or the manufacture of preparations in the form of a solid composition, according to the dosage form of the preparation.

The solid composition can be used as a material for producing such a preparation.

The content of the solid composition in the preparation of the present invention may vary depending on the dosage form of the preparation.

For example, the lower limit thereof may be 10, 20, 30, 40, 50, 60, 70, 80, 90, or 100 mass %.

For example, the upper limit thereof may be 20, 30, 40, 50, 60, 70, 80, 90, or 100 mass %.

The content may be, for example, 10 to 90 mass %, 20 to 80 mass %, 30 to 70 mass %, or 40 to 60 mass %.

The amount of the preparation (e.g., oral curcumin preparation) of the present invention to be administered or ingested may vary according to the age, body weight, and condition of the user; form of administration; treatment period; and the like. For example, the WHO Technical Report series shows that the ADI of curcumin is 0 to 3 mg/body weight (kg)/day, and that the NOAEL of curcumin is 250 to 320 mg/body weight (kg)/day (WHO Technical Report Series: 1237259778265_0.pdf, page 33). The preparation in an amount within this range can be preferably administered or ingested once a day, or in divided doses several times a day (e.g., twice, three times, four times, or five times).

The solid composition obtained by the present invention can be used not only for pharmaceutical products, foods, or the like, but can also be used by incorporation into, for example, cosmetics. Examples of the form of such cosmetics include skin care cosmetics such as lotions, creams, skin lotions, emulsions, and serums; hair care products, such as shampoos; mouthwashes; and the like. Further, any components that are commonly used in the field of cosmetics can be used in combination.

Examples of surfactants include anionic surfactants such as glycerol fatty acid esters, propylene glycol fatty acid esters, sorbitan fatty acid esters, polyoxyethylene alkyl ethers, polyoxyethylene alkyl phenyl ethers, polyoxyethylene fatty acid esters, carboxylic acid salts, and sulfonic acid salts; and cationic surfactants such as amine salts and ammonium salts. For example, at least one of such surfactants can be used in combination with the solid composition of the present invention.

When administered or ingested orally or via the gastrointestinal tract, the solid composition of the present invention exhibits a high ability to dissolve curcumin into the aqueous medium (e.g., body fluid such as gastric fluid, intestinal fluid, or saliva) in the body for a prolonged period of time, thus enabling efficient ingestion of the curcumin.

In other words, the composition of the present invention is exposed to the aqueous medium (e.g., body fluid such as gastric fluid, intestinal fluid, or saliva) in the body, and the curcumin contained in the composition is highly absorbed to the cells of the body via the medium.

The solid composition of the present invention may preferably be used to treat or prevent diseases or symptoms that benefit from curcumin absorption into cells.

In this specification, the term “treatment” may also mean alleviation of the symptoms that have already occurred.

In this specification, the term “prevention” may also mean alleviation of the symptoms that may occur.

The diseases or symptoms of the present invention may be at least one member selected from the group consisting of:
(1) diseases or symptoms that benefit from modulation of one or more transcription factors selected from the group consisting of NF-κB, AP-1, STAT (e.g., STAT-1, STAT-3, STAT-4, STAT-5), Wnt/β-catenin, Notch-1, EGR-1, CREB-BP, WT-1, HIF, ERE, Nrf-2, PPAR-α, and PPAR-γ;
(2) diseases or symptoms that benefit from modulation of one or more cytokines selected from the group consisting of TNF-α, IL-1β, IL-2, IL-5, IL-6, IL-8, IL-12, IL-18, MCP-1, MIP-1α, and MaIP;
(3) diseases or symptoms that benefit from modulation of one or more receptors selected from the group consisting of IR, ER-α, H2R, HER-2, LDLR, ITR, FasR, EPCR, AR, EGFR, IL-8R, CXCR4, AHR, and DR-5;
(4) diseases or symptoms that benefit from modulation of one or more enzymes selected from the group consisting of Desaturase, GCL, AATF-1, ATFase, Telomerase, MMP, ATPase, GICL, COX-2, iNOS, NQO-1, 5-LOX, TMMP-3, DNA pol, Src-2, FPT, PhP D, GST, ODC, and ACOX-1;
(5) diseases or symptoms that benefit from modulation of one or more growth factors selected from the group consisting of HGF, CTGF, FGF, NGF, PDGF, TGF-β1, EGF, VEGF, and TF;
(6) diseases or symptoms that benefit from modulation of one or more kinases selected from the group consisting of FAK, AAPK, P60c-tk, EGFR-K, Ca²⁺PK, PTK, MAPK, IL-1R AK, PKB, PKA, PAK, JAK, ERK, PhK, and JNK;
(7) diseases or symptoms that benefit from modulation of one or more members selected from the group consisting of uPA, Bcl-2, Bcl-xL, VCAM-1, ICAM-1, ELAM-1, IAP-1, Hsp-70, Cyclin D1, MDRP, p53, and DEF-40;
(8) diseases or symptoms that benefit from inhibition of aggregation of amyloid β;
(9) diseases or symptoms that benefit from suppression of aggregation of α-synuclein;
(10) diseases or symptoms that benefit from a decrease of one or more members selected from the group consisting of ALT, AST, and γ-GTP;
(11) diseases or symptoms that benefit from inhibition of HAT activity of p300; and
(12) diseases or symptoms that benefit from antioxidant effects.

The diseases or the symptoms of the present invention may be at least one member selected from the group consisting of:
(1) treatment or prevention of cancers (e.g., lung cancer, stomach cancer, colon cancer, liver cancer, pancreas cancer, breast cancer, prostate cancer) or tumors (e.g., malignant tumors) (including tumor shrinkage, inhibition of metastasis, and the like, as generally understood in the medical or pharmaceutical fields);
(2) treatment or prevention of diabetes;
(3) treatment or prevention of hyperglycemia;
(4) treatment or prevention of periodontitis;
(5) treatment or prevention of Alzheimer's disease or mild cognitive impairment;
(6) treatment or prevention of Parkinson's disease;
(7) treatment or prevention of nerve disorder;
(8) treatment or prevention of inflammation;
(9) treatment or prevention of amyloidosis;
(10) protection of liver function;
(11) treatment or prevention of heart failure;
(12) treatment or prevention of myocardial infarction;
(13) treatment or prevention of muscle fatigue;
(14) protection of kidney function;
(15) treatment or prevention of osteoporosis;
(16) treatment or prevention of depression;
(17) treatment or prevention of multiple sclerosis;
(18) treatment or prevention of ischemia; and
(19) treatment or prevention of hangover symptoms after alcohol ingestion.

Examples of the hangover symptoms after alcohol ingestion include nausea, headache, and stomach discomfort, as generally understood.

The treatment or prevention of the diseases or the symptoms of the present invention may be at least one member selected from the group consisting of: suppression of cholesterol elevation, suppression of triglyceride elevation, suppression of chylomicron elevation, suppression of blood pressure elevation, suppression of blood glucose elevation, anti-allergy, and suppression of body fat.

### 3. Method for Producing Solid Composition

The solid composition can be produced, for example, through a method comprising mixing:
(1) crystalline curcumin;
(2) hydrophilic polymer; and
(3) at least one nonionic surfactant selected from the group consisting of polyglycerol fatty acid esters, sucrose fatty acid esters, and lecithins;
   and adding
(4) other optional components,
   the method comprising the step of converting the crystalline curcumin to amorphous curcumin.

In the mixing step, the above components can be mixed simultaneously or successively.

The mixing step can be preferably performed without using a solvent such as an organic solvent.

Even when the solvent is used, the above components, such as curcumin, do not have to be completely dissolved in the solvent.

This allows the composition of the present invention to be produced at low cost, without using a large container or the like.

The step of mixing the components and the step of converting the crystalline curcumin to amorphous curcumin can be separate steps, or they can be partially or completely in common.

A higher conversion of crystalline curcumin to amorphous curcumin is preferable. Converting all or substantially all of the crystalline curcumin to amorphous curcumin is particularly preferable.

The solid composition can be produced, for example, by solvent precipitation methods, spray-drying methods, freeze-drying methods, drying under reduced pressure, or kneading methods; or a combination of these methods.

The solid composition is preferably produced by a production method comprising the step of kneading:
(1) crystalline curcumin;
(2) hydrophilic polymer;
(3) at least one nonionic surfactant selected from the group consisting of polyglycerol fatty acid esters, sucrose fatty acid esters, and lecithins; and
(4) other optional components.

In the kneading step, the crystalline curcumin, the hydrophilic polymer, and the nonionic surfactant are preferably kneaded simultaneously.

The kneading converts a part of the crystalline curcumin to amorphous curcumin, or preferably converts all or substantially all of the crystalline curcumin to amorphous curcumin.

The kneading can be preferably performed, for example, by using a single-screw extruder, an intermeshing screw extruder, or a multi-screw extruder (e.g., a twin-screw extruder). The kneading can also be preferably performed by kneading with a relatively weak force, such as kneading by hand using a spatula or the like on a hot plate.

In this kneading, for example, the mixture is kneaded while heated to the temperature at which the components are dissolved; then, after the components are dissolved, the mixture is cooled to room temperature. The resulting solid composition is pulverized into a powder using a pulverizer to obtain the composition of the present invention.

The primary particle diameter of the solid composition may be appropriately selected according to the form of the preparation of the present invention.

For example, the lower limit of the primary particle diameter of the solid composition may be 0.1 μm, 0.5 μm, 1 μm, 5 μm, 10 μm, 50 μm, or 100 μm.

For example, the upper limit of the primary particle diameter of the solid composition may be 0.5 μm, 1 μm, 5 μm, 10 μm, 50 μm, 100 μm, or 200 μm.

For example, the primary particle diameter may be in the range of 0.1 to 500 μm, 0.5 to 500 μm, 0.5 to 200 μm, 1 to 100 μm, or 10 to 100 μm.

In the preparation of the present invention, the primary particles of the solid composition may constitute secondary particles or the preparation itself according to the dosage form of the preparation, as can be generally understood by a person skilled in the art.

Further, in the preparation of the present invention, the solid composition may not be in the form of particles; and may be in the form of, for example, an uniform tablet, as can be generally understood by a person skilled in the art.

The solid composition is preferably produced by, for example, a method comprising the steps of:
fully mixing the crystalline curcumin, the hydrophilic polymer, and the nonionic surfactant with an oil or fat to prepare a slurry in which the curcumin is dissolved; and
drying the slurry.

Examples of drying methods include spray-drying methods, freeze-drying methods, vacuum-drying methods, drum-drying methods, far-infrared drying methods, and the like. Spray-drying methods are particularly preferable.

### EXAMPLES

The present invention is described in more detail below with reference to Examples. However, the scope of the present invention is not limited to these Examples.

The symbols and abbreviations in the Examples are defined as follows.
CUR: curcumin
PVP: polyvinylpyrrolidone
PGFE: polyglycerol fatty acid ester
HPC: hydroxypropyl cellulose
HPMC: hydroxypropyl methylcellulose

In the Examples, fine granules refer to fine-grained agents, as described in the Japanese Pharmacopoeia, 17th edition; i.e., a preparation of which the total amount passes through a No. 18 (850 μm) sieve, and of which 10% or less of the total amount remains on a No. 30 (500 μm) sieve.

In the Examples, “%” can be understood to mean mass %, based on common technical knowledge and the context, unless otherwise specified.

### Sample Preparation Method

Compositions having the formulations shown below in Table 1 were individually kneaded with heating to the melting temperature. After melting, each composition was cooled to room temperature, and formed into a powder using a pulverizer. The powder thus obtained was used. However, in the preparation of the composition of Comparative Example 3, the components were merely mixed without heating, and the resulting mixture was used as a test sample.

The kneading with heating was performed by setting a hot plate at 240° C., and kneading each composition by hand using a spatula or the like until the composition was melted.

The components used in the Examples or Comparative Examples are described below. Except for PGFE(A), commercially available products were purchased and used. PGFE(A) is a polyglycerol myristic acid ester of HLB12.

### Components

### Curcumin

Curcumin material (purity: containing 90% or more curcumin, 4% or more bisdemethoxycurcumin, and 0.1% or more demethoxycurcumin) (bulk powder)

### Hydrophilic Polymer

Kollidon K30 (trade name, BASF A.G.): PVP (polyvinylpyrrolidone)

### Nonionic Surfactants

PGFE(A): PGFE (polyglycerol fatty acid ester)

Ryoto Polyglyester 1-50SV (trade name, produced by Mitsubishi-Chemical Foods Corporation): PGFE (decaglycerol stearic acid ester)

Ryoto Polyglyester M-10D (trade name, Mitsubishi-Chemical Foods Corporation): PGFE (decaglycerol myristic acid ester)

NIKKOL HCO-60 (trade name, Nikko Chemicals Co., Ltd.): polyoxyethylene hydrogenated castor oil

NIKKOL TS-10V (trade name, Nikko Chemicals Co., Ltd.): polyoxyethylene sorbitan higher fatty acid ester (Polysorbate 60)

NIKKOL TO-10V (trade name, Nikko Chemicals Co., Ltd.): polyoxyethylene sorbitan higher fatty acid ester (Polysorbate 80)

NIKKOL TMGS-15V (trade name, Nikko Chemicals Co., Ltd.): polyoxyethylene glyceryl monostearate

TABLE 1

| | CUR | Hydrophilic | | | | |
|---|---|---|---|---|---|---|
| | material | polymer | | Nonionic surfactant | | |
| | (wt %) | Type | (wt %) | Type | (wt %) | Total |
| Example 1 | 11 | Kollidon | 64 | PGFE(A) | 25 | 100 |
| | | K30 | | | | |
| Example 2 | 11 | Kollidon | 64 | Ryoto Polyglyester M-10D | 25 | 100 |
| | | K30 | | | | |
| Example 3 | 11 | Kollidon | 64 | Ryoto Polyglyester 1-50SV | 25 | 100 |
| | | K30 | | | | |
| Example 4 | 14 | Kollidon | 76 | PGFE(A) | 10 | 100 |
| | | K30 | | | | |
| Example 5 | 7.5 | Kollidon | 42.5 | PGFE(A) | 50 | 100 |
| | | K30 | | | | |
| Example 6 | 11 | Kollidon | 64 | Ryoto Sugar Ester P-1560 | 25 | 100 |
| | | K30 | | | | |
| Example 7 | 11 | Kollidon | 64 | Ryoto Sugar Ester P-1670 | 25 | 100 |
| | | K30 | | | | |
| Example 8 | 11 | Kollidon | 64 | SLP White | 25 | 100 |
| | | K30 | | | | |
| Example 9 | 11 | Kollidon | 64 | Solec K-EML | 25 | 100 |
| | | K30 | | | | |
| Comparative | 15 | Kollidon | 85 | — | — | 100 |
| Example 1 | | K30 | | | | |
| Comparative | 30 | — | 0 | PGFE(A) | 70 | 100 |
| Example 2 | | | | | | |
| Comparative | 11 | Kollidon | 64 | PGFE(A) | 25 | 100 |
| Example 3 | | K30 | | | | |
| Comparative | 11 | Kollidon | 64 | NIKKOL HCO-60 | 25 | 100 |
| Example 4 | | K30 | | | | |
| Comparative | 11 | Kollidon | 64 | NIKKOL TO-10V | 25 | 100 |
| Example 5 | | K30 | | | | |
| Comparative | 11 | Kollidon | 64 | NIKKOL TO-10V | 25 | 100 |
| Example 6 | | K30 | | | | |
| Comparative | 11 | Kollidon | 64 | NIKKOL TMGS-15V | 25 | 100 |
| Example 7 | | K30 | | | | |

X-ray diffraction measurement showed that the peak of crystalline curcumin disappeared completely or partially in Examples 1 to 9, Comparative Example 1, and Comparative Examples 4 to 7; and confirmed that the compositions obtained in these Examples and Comparative Examples contained amorphous curcumin. Differential scanning calorimetry showed that the peak of crystalline curcumin was reduced in Comparative Example 2, and the results confirmed that the composition obtained in Comparative Example 2 contained amorphous curcumin. Since the composition of Comparative Example 3 is merely a mixture of the components, the composition is assumed to contain crystalline curcumin.

### Dissolution Test Method

A dissolution test was performed in accordance with the test method described in the Japanese Pharmacopoeia, 16th edition using the following materials under the following conditions. Analysis was performed by sampling a small amount of each test liquid at various times.

### Materials and Conditions for the Dissolution Test

### [1] Curcumin Dissolution Test

Dissolution tester: PJ-32S (product name, produced by Miyamoto Riken Ind. Co., Ltd.)
Test liquid: Japanese Pharmacopoeia Second Fluid (artificial intestinal fluid, pH 6.8)
Sample amount: 10 mg/100 ml in terms of curcumin
Temperature: 37° C.
Sampling: After filtration through a 0.2-μm membrane, the filtrate was analyzed.
Analysis: the HPLC method

The test results are shown below.

### Test Example 1

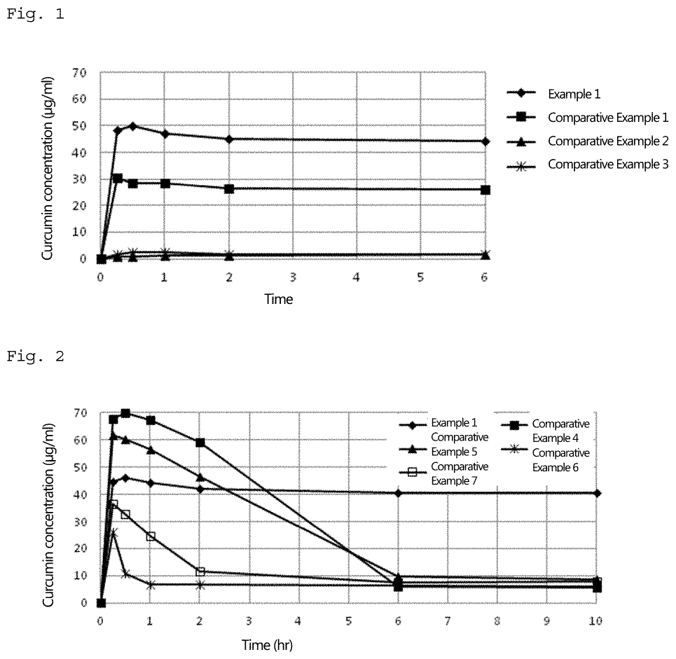

Samples shown in Table 2 were used to test the changes over time of curcumin dissolution into artificial intestinal fluid, in comparison with a solid composition containing no surfactants, a solubilized preparation, and a solid composition prepared without heating. Table 3 and FIG. 1 show the results. As can be understood from these results, the composition of the present invention exhibited high ability to dissolve curcumin into body fluids (preferably intestinal fluid), and this was maintained for a prolonged period of time.

TABLE 2

| | Formulation | CUR material:PVP | CUR material:PGFE | |
|---|---|---|---|---|
| | (Numerical values | (Numerical values | (Numerical values | |
| Sample | are by wt %.) | indicate mass ratios.) | indicate mass ratios.) | Remarks |
| Example 1 | CUR material: 11/ | 15:85 | 30:70 | Amorphous |
| | PVP: 64/PGFE(A): 25 | | | preparation |
| | | | | (containing |
| | | | | PGFE) |
| Comparative | CUR material: 15/ | 15:85 | — | Amorphous |
| Example 1 | PVP: 85 | | | preparation (not |
| | | | | containing PGFE) |
| Comparative | CUR material: 30/ | — | 30:70 | Solubilized |
| Example 2 | PGFE(A): 70 | | | preparation |
| Comparative | CUR material: 11/ | 15:85 | 30:70 | The same |
| Example 3 | PVP: 64/PGFE(A): 25 | | | formulation as |
| | | | | in Example 1, |
| | | | | but without |
| | | | | heating |

TABLE 3

| | Curcumin concentration (μg/ml) at various times (hr) | | | | | |
|---|---|---|---|---|---|---|
| Sample | 0 hr | 0.25 hr | 0.5 hr | 1 hr | 2 hr | 6 hr |
| Example 1 | 0 | 48.1 | 49.9 | 47.0 | 44.9 | 44.2 |
| Comparative | 0 | 30.4 | 28.3 | 28.6 | 26.3 | 25.8 |
| Example 1 | | | | | | |
| Comparative | 0 | 0.8 | 0.9 | 1.0 | 1.2 | 1.6 |
| Example 2 | | | | | | |
| Comparative | 0 | 1.7 | 2.3 | 2.3 | 1.7 | 1.5 |
| Example 3 | | | | | | |

### Test Example 2

Samples shown in Table 4 were used to test the changes over time of curcumin dissolution into artificial intestinal fluid, in comparison with other nonionic surfactants. The results are shown in Table 5 and FIG. 2. As can be understood from these results, the composition of the present invention exhibited high ability to dissolve curcumin into body fluids (preferably intestinal fluid) only when a specific nonionic surfactant was used, and this was maintained for a prolonged period of time.

TABLE 4

| | | | CUR Material:Nonionic | |
|---|---|---|---|---|
| | Composition | CUR material:PVP | surfactant | |
| | (Numerical values | (Numerical values | (Numerical values | |
| Sample | are by wt.) | indicate mass ratios.) | indicate mass ratios.) | Remarks |
| Example 1 | CUR material: 11/ | 15:85 | 30:70 | PGFE |
| | PVP: 64/PGFE(A): 25 | | | |
| Comparative | CUR material: 11/ | 15:85 | 30:70 | Polyoxyethylene |
| Example 4 | PVP: 64/HCO60: 25 | | | hydrogenated |
| | | | | castor oil |
| Comparative | CUR material: 11/ | 15:85 | 30:70 | Polysorbate 60 |
| Example 5 | PVP: 64/TS10V: 25 | | | |
| Comparative | CUR material: 11/ | 15:85 | 30:70 | Polysorbate 80 |
| Example 6 | PVP: 64/TO10V: 25 | | | |
| Comparative | CUR material: 11/ | 15:85 | 30:70 | Polyoxyethylene |
| Example 7 | PVP: 64/TMGS15V: 25 | | | glyceryl |
| | | | | monostearate |

TABLE 5

| | Curcumin concentration (μg/ml) at various times (hr) | | | | | | |
|---|---|---|---|---|---|---|---|
| Sample | 0 hr | 0.25 hr | 0.5 hr | 1 hr | 2 hr | 6 hr | 10 hr |
| Example 1 | 0 | 44.7 | 46.3 | 44.5 | 42.1 | 40.6 | 40.7 |
| Comparative | 0 | 67.5 | 70.0 | 67.5 | 59.0 | 6.1 | 5.4 |
| Example 4 | | | | | | | |
| Comparative | 0 | 61.9 | 60.4 | 56.4 | 46.7 | 9.6 | 8.7 |
| Example 5 | | | | | | | |
| Comparative | 0 | 25.9 | 10.8 | 6.8 | 6.6 | 6.3 | 5.9 |
| Example 6 | | | | | | | |
| Comparative | 0 | 36.5 | 32.8 | 24.8 | 11.6 | 7.5 | 7.8 |
| Example 7 | | | | | | | |

### Test Example 3

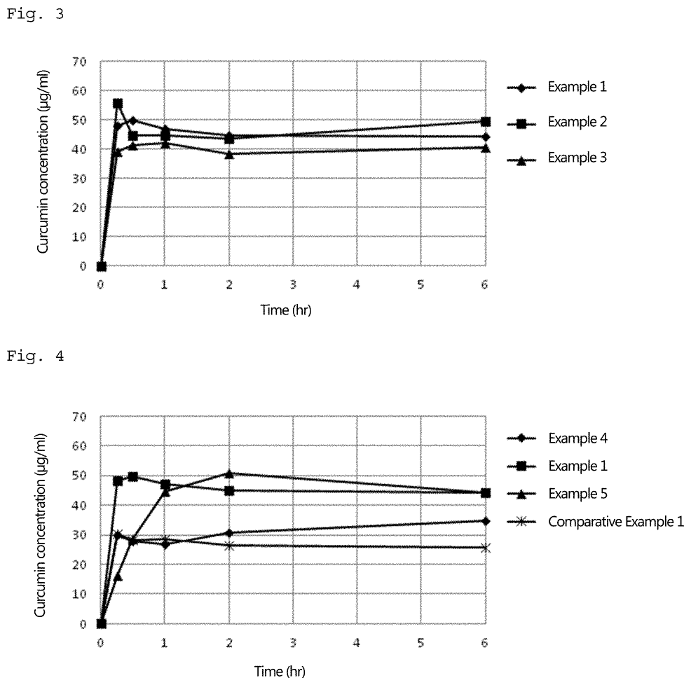

Samples shown in Table 6, which comprised various types of polyglycerol fatty acid esters, were used to test the changes over time of curcumin dissolution into artificial intestinal fluid. The results are shown in Table 7 and FIG. 3. As can be understood from these results, even when various types of polyglycerol fatty acid esters were used, the composition of the present invention exhibited high ability to dissolve curcumin into body fluids (preferably intestinal fluid), and this was maintained for a prolonged period of time.

TABLE 6

| | Composition | CUR material:PVP | CUR material:PGFE | |
|---|---|---|---|---|
| | (Numerical values | (Numerical values | (Numerical values | |
| Sample | are by wt. %) | indicate mass ratios.) | indicate mass ratios.) | Remarks |
| Example 1 | CUR Material: 11/ | 15:85 | 30:70 | PGFE (A) |
| | PVP: 64/PGFE(A): 25 | | | |
| Example 2 | CUR Material: 11/ | 15:85 | 30:70 | PGFE |
| | PVP: 64/M-10D: 25 | | | M-10D |
| Example 3 | CUR Material: 11/ | 15:85 | 30:70 | PGFE |
| | PVP: 64/1-50SV: 25 | | | 1-50SV |

TABLE 7

| | Curcumin concentration (μg/ml) at various times (hr) | | | | | |
|---|---|---|---|---|---|---|
| Sample | 0 hr | 0.25 hr | 0.5 hr | 1 hr | 2 hr | 6 hr |
| Example 1 | 0 | 48.1 | 49.9 | 47.0 | 44.9 | 44.2 |
| Example 2 | 0 | 55.9 | 44.9 | 44.8 | 43.8 | 49.5 |
| Example 3 | 0 | 39.1 | 41.4 | 42.3 | 38.3 | 40.6 |

### Test Example 4

Samples shown in Table 8, which comprised various amounts of polyglycerol fatty acid ester, were used to test the changes over time of curcumin dissolution into artificial intestinal fluid. The results are shown in Table 9 and FIG. 4. As can be understood from these results, even when the amount of polyglycerol fatty acid ester used varied, the composition of the present invention exhibited high ability to dissolve curcumin into body fluids (preferably intestinal fluid), and this was maintained for a prolonged period of time.

TABLE 8

| | Formulation | CUR material:PVP | CUR material:PGFE | |
|---|---|---|---|---|
| | (Numerical values | (Numerical values | (Numerical values | |
| Sample | are by wt. %) | indicate mass ratios.) | indicate mass ratios.) | Remarks |
| Example 1 | CUR material: 11/ | 15:85 | 30:70 | |
| | PVP: 64/PGFE(A): 25 | | | |
| Example 4 | CUR material: 14/ | 15:85 | 58:42 | A different |
| | PVP: 76/PGFE(A): 10 | | | amount of PGFE |
| | | | | was used |
| | | | | together. |
| Example 5 | CUR material: 7.5/ | 15:85 | 13:87 | A different |
| | PVP: 42.5/PGFE(A): 50 | | | amount of PGFE |
| | | | | was used |
| | | | | together. |
| Comparative | CUR material: 15/ | 15:85 | 100:0 | No PGFE was |
| Example 1 | PVP: 85 | | | used. |

TABLE 9

| | Time (hr) and curcumin concentration (μg/ml) | | | | | |
|---|---|---|---|---|---|---|
| Sample | 0 hr | 0.25 hr | 0.5 hr | 1 hr | 2 hr | 6 hr |
| Example 4 | 0 | 30.0 | 27.7 | 26.9 | 30.6 | 34.7 |
| Example 1 | 0 | 48.1 | 49.9 | 47.0 | 44.9 | 44.2 |
| Example 5 | 0 | 16.2 | 28.4 | 44.6 | 50.7 | 44.1 |
| Comparative | 0 | 30.4 | 28.3 | 28.6 | 26.3 | 25.8 |
| Example 1 | | | | | | |

### Test Example 5

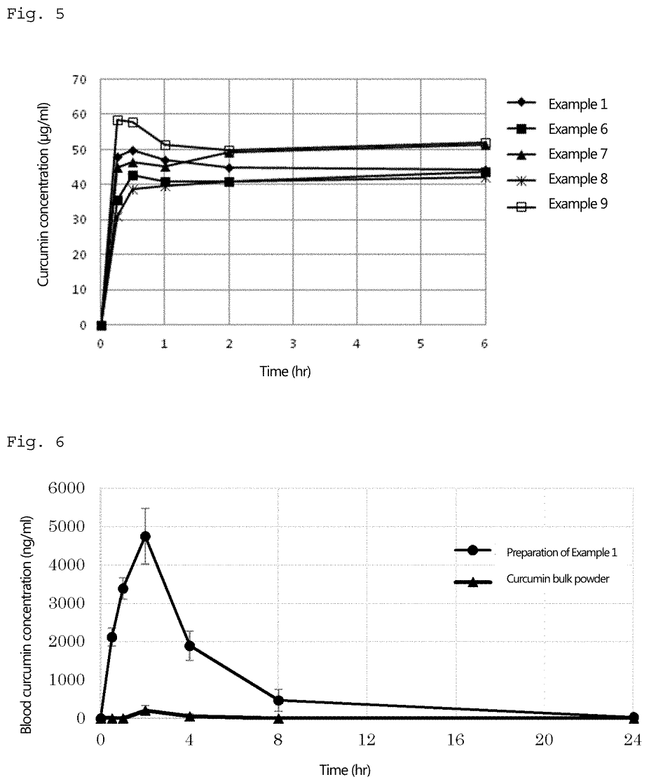

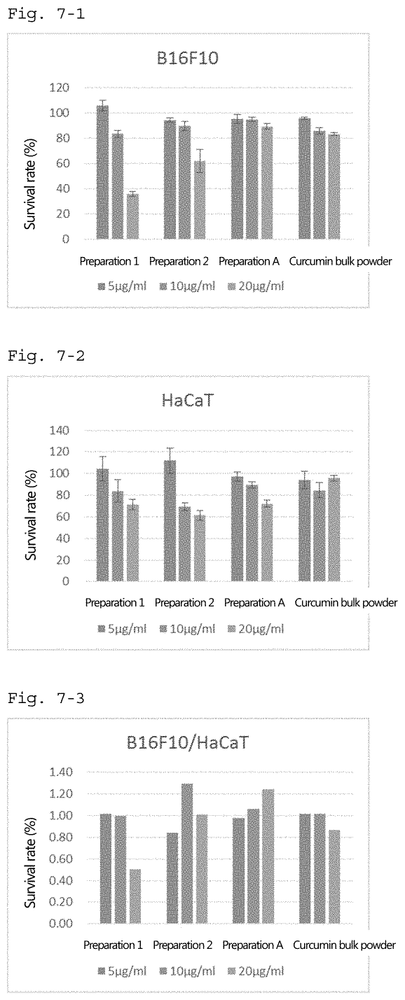

Samples shown in Table 10 were used to test the changes over time of curcumin dissolution into artificial intestinal fluid from compositions prepared using a sugar ester or lecithin, in comparison with the composition prepared using a polyglycerin fatty acid ester. The results are shown in Table 11 and FIG. 5. As can be understood from these results, even when a sugar ester or lecithin was used, the composition of the present invention exhibited high ability to dissolve curcumin into body fluids (preferably intestinal fluids) as in the case of using a polyglycerol fatty acid ester, and this was maintained for a prolonged period of time.

TABLE 10

| | | | CUR Material:Nonionic | |
|---|---|---|---|---|
| | Formulation | CUR Material:PVP | surfactant | |
| | (Numerical values | (Numerical values | (Numerical values | |
| Sample | are by wt. %.) | indicate mass ratios.) | indicate mass ratios.) | Remarks |
| Example 1 | CUR Material: 11/ | 15:85 | 30:70 | |
| | PVP: 64/PGFE(A): 25 | | | |
| Example 6 | CUR Material: 11/ | 15:85 | 30:70 | Sucrose |
| | PVP:4/P-1560: 25 | | | fatty |
| | | | | acid |
| | | | | ester |
| Example 7 | CUR Material: 11/ | 15:85 | 30:70 | Sucrose |
| | PVP: 64/P-1670: 25 | | | fatty |
| | | | | acid |
| | | | | ester |
| Example 8 | CUR Material: 11/ | 15:85 | 30:70 | Soybean |
| | PVP: 64/SLP: 25 | | | lecithin |
| Example 9 | CUR Material: 11/ | 15:85 | 30:70 | Enzyme- |
| | PVP: 64/K-EML: 25 | | | treated |
| | | | | soybean |
| | | | | lecithin |

TABLE 11

| | Time (hr) and curcumin concentration (μg/ml) | | | | | |
|---|---|---|---|---|---|---|
| Sample | 0 hr | 0.25 hr | 0.5 hr | 1 hr | 2 hr | 6 hr |
| Example 1 | 0 | 48.1 | 49.9 | 47.0 | 44.9 | 44.2 |
| Example 6 | 0 | 35.7 | 42.7 | 41.0 | 40.8 | 43.7 |
| Example 7 | 0 | 44.8 | 46.5 | 45.0 | 49.3 | 51.2 |
| Example 8 | 0 | 31.1 | 38.8 | 39.6 | 41.0 | 42.0 |
| Example 9 | 0 | 58.5 | 57.7 | 51.4 | 49.9 | 51.9 |

### Test Example 6

Compositions were prepared using HPC or HPMC in place of PVP in accordance with the production method described above, and subjected to the same tests as described above. The results showed that the compositions prepared using HPC or HPMC exhibited low ability to dissolve curcumin into body fluids (preferably intestinal fluid), as compared with the composition prepared using PVP; however, the same tendency as the composition using PVP was confirmed, and the dissolution of curcumin into body fluids (preferably intestinal fluid) was maintained for a prolonged period of time.

### Test Example 7

The changes over time of blood curcumin concentration in rats to which the amorphous preparation of Example 1 was administered was investigated by the following test method. As a comparative example, a bulk curcumin powder was administered.

### Test Method

Animals: Three SD rats (male, 7 weeks old, fasted for 14 to 16 hours before administration) per group were used.

Administration: 100 mg/kg (in terms of curcumin), single oral administration (sonde method)

Blood sampling: Jugular venous blood sampling immediately before administration; and 0.5, 1, 2, 4, 8, and 24 hours after administration

Analysis: 25 μl of plasma was enzymatically treated with β-glucuronidase. After extraction of curcumin with acetonitrile, the solvent was evaporated to dryness. The resulting product was rediluted with methanol, and measured by UV detection (420 nm).

FIG. 6 shows a graph of the analysis results. The results confirmed that when the preparation of the present invention is used, poorly water-soluble curcumin can be highly absorbed in the living body over a prolonged period of time.

### Test Example 8

### Cytotoxicity Test

In Test Example 8, the following preparations were used as samples.

Preparations 1 to 3 were produced in the same manner as the preparation of Example 1, except that the mixing ratio of the components was changed.
Preparation 1 (CUR material:PVP:PGFE (A)=16:49:35)
Preparation 2 (CUR material:PVP:PGFE (A)=25:75:0)
Preparation 3 (CUR material:PVP:PGFE (A)=11:64:25)
Preparation A (a curcumin preparation produced according to the method described in Example 24 of Japanese Patent No. 5448511) (containing a curcumin bulk powder (powder that passed through a 80 mesh sieve, and containing 90% or more curcumin, 4% or more bisdemethoxycurcumin, and 0.1% or more demethoxycurcumin) and dextrin)

### Test Example 8-1

### Skin Cancer Cells (1)

A cytotoxicity test was performed using the following samples under the following conditions, by the following methods.

### Method

Samples were diluted with PBS (phosphate-buffered physiological saline) to 3 mg/ml in terms of curcumin to prepare diluted samples.

Cells were seeded in a medium. After 24 hours of culture, the diluted samples were added in predetermined amounts. After 24 hours of culture, a WST8 reagent was added, and absorbance was measured.

### Cell Culturing Conditions

Cells: B16F10 (skin cancer cells (metastatic cells)), HaCaT (human epidermal keratinocytes)

Medium: DMEM (high glucose), 10% FCS, 1% Ab

Culturing conditions: 37° C., 5% CO₂, 24 hours

### Samples

Samples: Preparation 1, Preparation 2, Preparation A, and Curcumin bulk powder.

Concentration of each sample added: 5, 10, and 20 μg/ml (in terms of curcumin).

FIGS. 7-1 to 7-3 show the test results. Three columns of the bar graph for each sample, from left to right, in each figure show the results of the preparations to which each sample was added in an amount of 5, 10, and 20 μg/ml (in terms of curcumin).

As can be understood from these results, the preparation of the present invention showed a concentration-dependent efficacy in killing skin cancer cells in the tested concentration range, and acted more strongly on skin cancer cells than on normal cells.

This indicates that curcumin in the preparation of the present invention is readily absorbed by cells; and that the preparation of the present invention is effective for treating tumors, and has fewer side effects.

### Test Example 8-2

### Breast Cancer Cells

A cytotoxicity test was performed in the same manner as in Test Example 8-1, except that the following cells and samples were used.
Cells: MDA-MB-436 (breast cancer cells)
Samples: Preparation 3 and Preparation A
Concentration of each sample added: 3.8, 7.5, 15, and 30 μg/ml (in terms of curcumin).

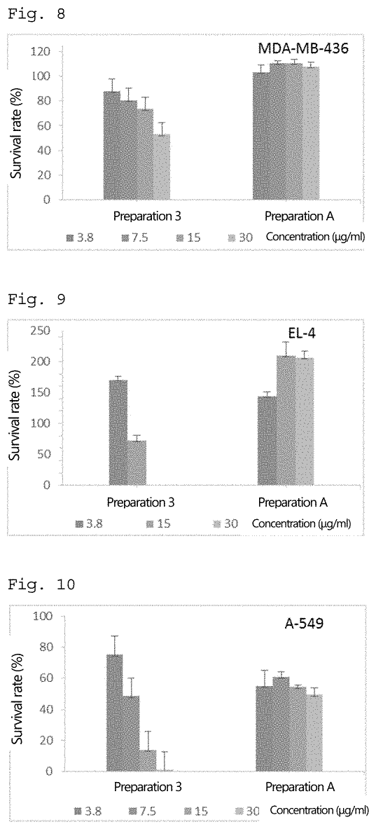

FIG. 8 shows the test results. As can be understood from the results, the preparation of the present invention showed a concentration-dependent efficacy in killing breast cancer cells in the tested concentration range.

This indicates that curcumin in the preparation of the present invention is readily absorbed by cells, and that the preparation of the present invention is effective for treating tumors.

### Test Example 8-3

### Lymphoma Cells

A cytotoxicity test was performed in the same manner as in Test Example 8-1, except that the following cells and samples were used.
Cells: EL-4 (lymphoma cells)
Samples: Preparation 3 and Preparation A
Concentration of each sample added: 7.5, 15, and 30 μg/ml (in terms of curcumin)

FIG. 9 shows the test results. As can be understood from the results, the preparation of the present invention showed efficacy in killing lymphoma cells in the tested concentration range.

This indicates that curcumin in the preparation of the present invention is easily absorbed by cells, and that the preparation of the present invention is effective for treating tumors.

### Test Example 8-4

### Lung Cancer Cells

A cytotoxicity test was performed in the same manner as in Test Example 8-1, except that the following cells and samples were used.
Cells: A-549 (lung cancer cells)
Samples: Preparation 3 and Preparation A
Concentration of each sample added: 3.8, 7.5, 15, and 30 μg/ml (in terms of curcumin)

FIG. 10 shows the test results. As can be understood from the results, the preparation of the present invention showed a concentration-dependent efficacy in killing lung cancer cells in the tested concentration range.

This indicates that curcumin in the preparation of the present invention is readily absorbed by cells, and that the preparation of the present invention is effective for treating tumors.

### Test Example 8-4

### Skin Cancer Cells (2)

A cytotoxicity test was performed using the following samples under the following conditions, by the following method.

### Method

A LDH-Cytotoxic Test Wako kit was used for this test. Samples were diluted with PBS to 3 mg/ml in terms of curcumin to prepare diluted samples.

Cells were seeded in a medium. After 24 hours of culture, the diluted samples were added in predetermined amounts.

After 24 hours of culture, a coloring reagent was added, and the cells were allowed to stand for 45 minutes. Absorbance (570 nm) was measured within 90 min after adding a reaction-stop solution.

### Cell Culturing Conditions

Cell: B16F10 (skin cancer cells (metastatic cells))

Medium: DMEM (high glucose), 10% FCS, 1% Ab

Culture conditions: 37° C., 5% CO₂, 24 hours

### Samples

Preparation 3 and Preparation A

Concentration of sample added: 30 μg/ml (in terms of curcumin)

Coloring reagents: nitroblue tetrazolium, diaphorase, NAD

Reaction-stop reagent: hydrochloric acid (1 mol/L)

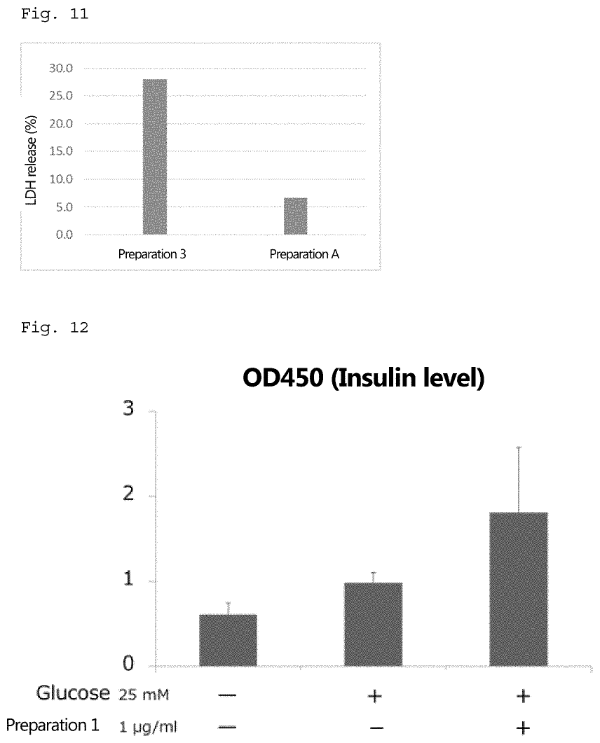

FIG. 11 shows the test results. A higher LDH release means more impaired B16F10. As can be understood from the results, the preparation of the present invention has shown efficacy in killing skin cancer cells in the tested concentration range.

This indicates that curcumin in the preparation of the present invention is readily absorbed by cells, and that the preparation of the present invention is effective for treating tumors.

### Test Example 9

An insulin secretion test was performed using MIN6 cells (mouse pancreatic β cells) and the following samples under the following conditions, by the following method.

### Method

Samples were diluted with PBS to 3 mg/ml in terms of curcumin to prepare diluted samples.

Cells were seeded in a medium, and the diluted samples were added.

After 24 hours of culture, the cells were washed 3 times with KRBH buffer (0 mM glucose), and then incubated with KRBH buffer (0 mM glucose) for 1 hour.

Further, after washing once with KRBH buffer (0 mM glucose), the cells were incubated with KRBH buffer (25 mM glucose) for 24 hours.

The supernatant was collected, and insulin levels were measured by ELISA. (Absorbance measured at 450 nm.)

### Cell Culturing Conditions

Cells: MIN6 cells (Mouse Pancreatic β Cells)

Media: DMEM (high glucose), 10% FCS, 1% Ab, 70 μM 2-ME

Culture conditions: 37° C., 5% CO₂

### Sample

Preparation 1

Concentration of the sample added: 1 μg/ml (in terms of curcumin)

FIG. 12 shows the test results.

As can be understood from the results, the preparation of the present invention increased insulin secretion in MIN6 cells (mouse pancreatic β-cells) by adding glucose.

The results show that curcumin in the preparation of the present invention is readily absorbed by cells, and that the preparation of the present invention is effective for treating diabetes.

### Test Example 10

Acute toxicity tests (organ weight measurement, biochemical testing, and blood cell testing) were performed using the following samples under the following conditions, by the following method.

### Method

The following samples were individually administered to the tail vein of mice (BALB/c). After 24 hours, the mice were dissected and subjected to organ weight measurement, biochemical testing, and blood cell testing. The biochemical testing was performed by measurement with a Fuji Dri-Chem, and the blood cell testing was performed by measurement with an XT-2000i multi-item automatic blood cell analyzer.

### Sample

### Preparation 3

Amounts added: 0.25 mg/kg, 5 mg/kg, and 100 mg/kg (in terms of curcumin)

5 mg/kg is an amount that is almost equivalent to the ADI defined by WHO, and 100 mg/kg is about 30 times the ADI defined by WHO.

### NT: PBS

Control: containing all of the components of Preparation 3 other than curcumin

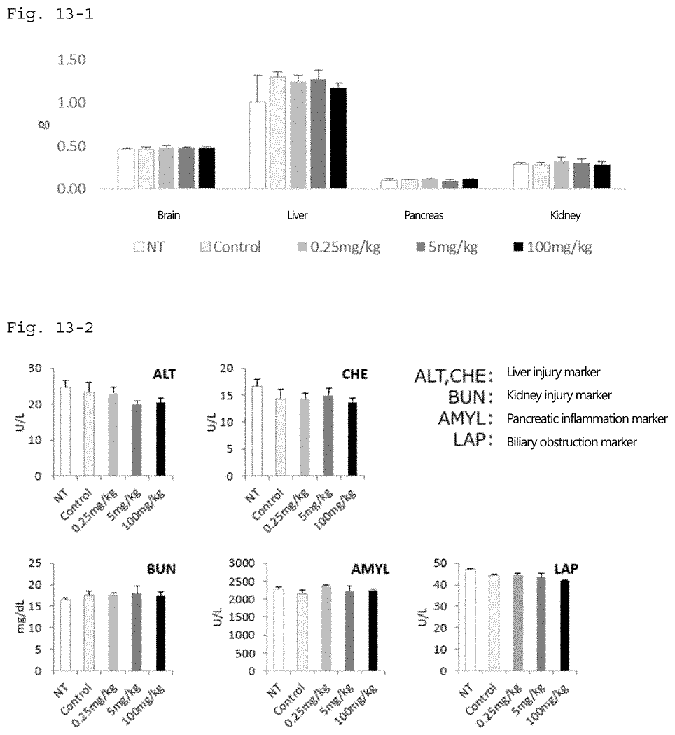

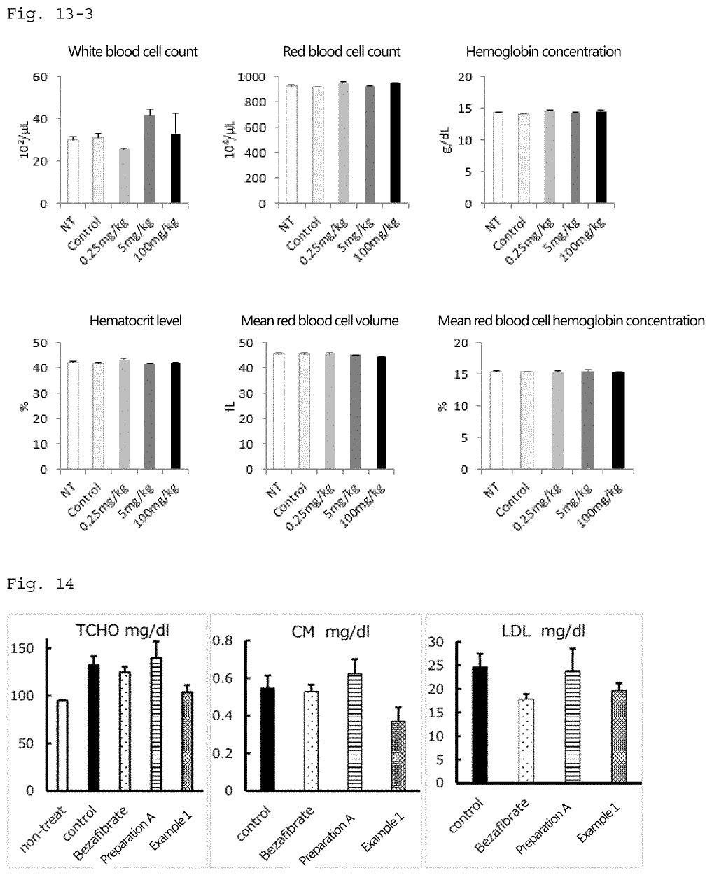

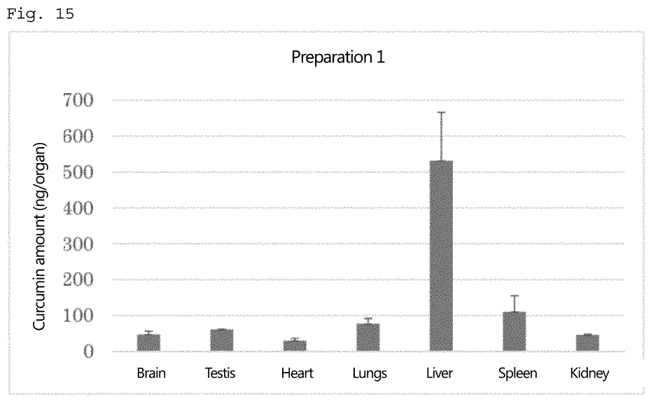

FIGS. 13-1 to 13-3 show the test results of organ weight measurement, biochemical testing, and blood cell testing, respectively.

As can be understood from these results, even when curcumin was overdosed, the preparation of the present invention did not bring about any acute toxicity that would affect organ weight, biochemical markers, or blood cells.

### Example 10

### Preparation Production Example

A preparation comprising a CUR material, Kollidon K30, and PGFE (A) at a mixing ratio of 16:49:35 was produced by the same production method as for the preparation of Example 1.

### Test Example 11

### Curcumin Administration Test (A) (TCHO, CM, LDL) in HFD-Challenged Mice

A curcumin administration test was performed using HFD (high-fat diet)-challenged mice and the following samples under the following conditions, by the following methods.

In this test, total cholesterol (TCHO), chylomicron (CM), and LDL cholesterol (LDL) were measured.

### Method

Mice (C57BL/6, 5 weeks old, male, 6 to 7 mice per group) were allowed to freely drink an aqueous liquid of one of the samples described below, together with a high-fat diet (however, for the non-treated group described below, a normal diet) for 12 weeks, and evaluated for various parameters.

This test was performed using the test groups described below.

The following materials were given to the test groups.

### Materials

### (1) Feed

Normal diet: D12450B (Research Diets)

High-fat diet: D12451 (Research Diets)

### (2) Ingested Sample

Bezafibrate: a 0.081 mass % aqueous Bezafibrate (Wako Pure Chemical Industries) liquid

Preparation A: a 0.04 mass % (in terms of curcumin) aqueous liquid of Preparation A (fine granules)

Example 1: a 0.04 mass % (in terms of curcumin) aqueous liquid of the preparation of Example 1

### Test Groups

Non-treated group: normal diet and water
Control group: high-fat diet and water (by ad libitum drinking)
Bezafibrate group: high-fat diet and aqueous Bezafibrate liquid (by ad libitum drinking)
Preparation A group: high-fat diet and aqueous liquid of Preparation A (by ad libitum drinking)
Example 1 group: high-fat diet and aqueous liquid of the preparation of Example 1 (by ad libitum drinking)

### Method for Analyzing Total Cholesterol, Chylomicron, and LDL Cholesterol Concentrations in Plasma

After the feeding period, blood was sampled and centrifuged at 3000G at 4° C. for 15 minutes to obtain plasma.

The plasma was measured by a Fuji Dri-Chem 4000V biochemical automatic analyzer (Fujifilm, Tokyo, Japan).

The results are shown in Tables 12 to 14, and FIG. 14.

As can be understood from these results, it became clear that the preparation of Example 1 has an inhibitory effect on the elevation of THO, CM, and LDL.

TABLE 12

| | Non- | | | Preparation | Example |
|---|---|---|---|---|---|
| | treated | Control | Bezafibrate | A | 1 |
| TCHO mg/dl | 94.8 | 132.0 | 125.0 | 140.0 | 103.8 |
| Standard | 1.1 | 9.6 | 5.9 | 17.3 | 7.6 |
| error | | | | | |

TABLE 13

| | | | Preparation | Example |
|---|---|---|---|---|
| | Control | Bezafibrate | A | 1 |
| CM mg/dl | 0.55 | 0.53 | 0.62 | 0.37 |
| Standard | 0.101 | 0.034 | 0.094 | 0.098 |
| error | | | | |

TABLE 14

| | | | Preparation | Example |
|---|---|---|---|---|
| | Control | Bezafibrate | A | 1 |
| LDL mg/dl | 24.7 | 17.9 | 23.9 | 19.7 |
| Standard | 2.8 | 1.1 | 4.7 | 1.6 |
| error | | | | |

### Test Example 12

### Biodistribution Evaluation

The curcumin biodistribution after administration of curcumin preparations was evaluated under the following conditions, by the following methods.

### Method

Test samples were orally administered to rats (SD rats, 7 weeks old, male, fasted for 14 to 16 hours before administration, 3 mice per group), and the curcumin concentration in each organ was analyzed after 24 hours.

Dosage: 100 mg/kg in terms of curcumin

Administration method: single oral administration (sonde method)

### Analysis Method

Twenty-four hours after the administration, the rats were perfused with 50 ml of PBS or more to remove blood, and then dissected to remove organs.

Each organ was homogenized with 4 ml of 0.1% formic acid methanol per gram of the organ, and 500 μl of the homogenate was centrifuged at 10000G for 10 minutes to obtain a supernatant.

After the supernatant was evaporated to dryness under nitrogen and re-diluted with 80% methanol, the diluted supernatant was analyzed by UV detection (420 nm).

The results are shown in Tables 15 to 16, and FIG. 15.

TABLE 15

| | (ng/organ) | | | | | | |
|---|---|---|---|---|---|---|---|
| | Brain | Testis | Heart | Lung | Liver | Kidney | Spleen |
| Preparation 1 | 47.4 | 61.3 | 30.1 | 77.0 | 531.8 | 110.4 | 45.9 |
| Preparation A | 48.3 | 81.2 | 35.9 | 69.3 | 243.9 | 129.8 | 44.2 |

TABLE 16

| | (Standard error) (ng/organ) | | | | | | |
|---|---|---|---|---|---|---|---|
| | Brain | Testis | Heart | Lung | Liver | Kidney | Spleen |
| Preparation 1 | 9.2 | 0.7 | 6.5 | 14.6 | 134.5 | 44.7 | 1.9 |
| Preparation A | 2.3 | 13.9 | 13.2 | 23.7 | 91.9 | 61.9 | 0.7 |

### Test Example 13

### Evaluation of Tissue Distribution by Long-Term Ingestion (After 3 Months of Administration by Ingestion)

The tissue distribution after long-term ingestion of the preparation of the present invention was evaluated under the following conditions, by the following method.

### Test Method

Mice (BALB/c mice, 6 weeks old, male, 4 mice per group) were allowed to freely drink an aqueous liquid (concentration: 0.1% in terms of curcumin) of a curcumin preparation (preparation A or the preparation of Example 1) for 3 months, and the curcumin concentration in each organ was then analyzed.

After the mice were allowed to freely drink an aqueous solution of a curcumin preparation under the same conditions as above for 3 months, and then allowed to drink water in place of the aqueous solution for 24 hours, the curcumin concentration in each organ was analyzed.

### Analysis Method

After 3 months of the drinking, the mice were perfused with 5 ml of PBS or more to remove blood, and then dissected to remove organs.

Each organ was homogenized with 4 ml of 0.1% formic acid methanol per gram of the organ.

500 μl of each homogenate was centrifuged at 10000G for 10 minutes to obtain a supernatant.

After the supernatant was evaporated to dryness under nitrogen and re-diluted with 80% methanol, the diluted supernatant was measured by UV detection (420 nm).

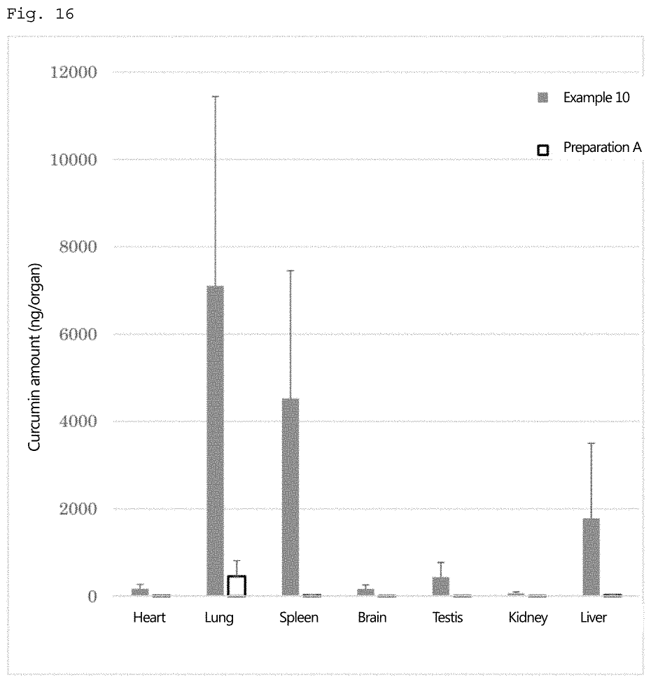

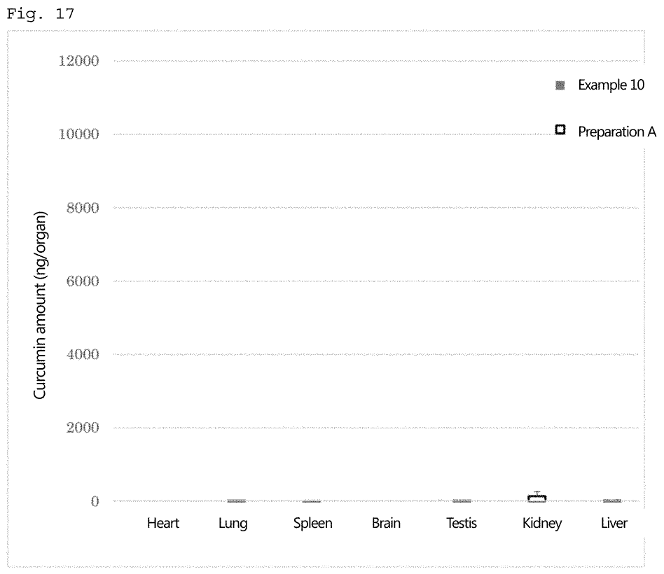

The results are shown in Tables 17 to 20, and FIGS. 16 and 17.

TABLE 17
Curcumin amount after 3 months of ingestion

| | (ng/organ) | | | | | | |
|---|---|---|---|---|---|---|---|
| | Heart | Lung | Spleen | Brain | Testis | Kidney | Liver |
| Example 10 | 170.2 | 7104.8 | 4524.8 | 164.7 | 434.3 | 62.3 | 1778.7 |
| Preparation | 10.8 | 453.3 | 19.8 | 2.2 | 5.3 | 1.2 | 22.1 |
| A | | | | | | | |

TABLE 18
Standard error in curcumin amount after 3 months of ingestion

| | (ng/organ) | | | | | | |
|---|---|---|---|---|---|---|---|
| | Heart | Lung | Spleen | Brain | Testis | Kidney | Liver |
| Example 10 | 100.8 | 4335.0 | 2930.1 | 94.9 | 339.2 | 38.3 | 1721.1 |
| Preparation | 10.0 | 358.9 | 9.3 | 1.2 | 3.8 | 0.3 | 13.1 |
| A | | | | | | | |

TABLE 19
Curcumin amount after 24 hours of drinking

| | (ng/organ) | | | | | | |
|---|---|---|---|---|---|---|---|
| | Heart | Lung | Spleen | Brain | Testis | Kidney | Liver |
| Example 10 | 0.0 | 7.7 | 8.5 | 0.0 | 12.9 | 4.1 | 8.2 |
| Preparation A | 0.0 | 12.3 | 6.6 | 0.0 | 14.3 | 137.1 | 19.8 |

TABLE 20
Standard error in curcumin amount after 24 hours of drinking

| | (ng/organ) | | | | | | |
|---|---|---|---|---|---|---|---|
| | Heart | Lung | Spleen | Brain | Testis | Kidney | Liver |
| Example 10 | 0.0 | 3.2 | 6.4 | 1.9 | 7.7 | 1.4 | 3.7 |
| Preparation A | 0.0 | 1.5 | 0.3 | 0.9 | 5.2 | 117.7 | 6.8 |

### Test Example 14

### Curcumin Administration Test (B) in HFD-Challenged Mice

A curcumin administration test was performed using HFD (high-fat diet)-challenged mice and the following samples under the following conditions, by the following method.

In this test, triglyceride (TG) was measured.

### Test Method

Mice (C57BL/6, 5 weeks old, male, 8 to 9 mice per group) were allowed to freely drink an aqueous liquid of the sample described below, together with a high-fat diet (however, for mice in the non-treated group described below, a normal diet) for 12 weeks, and evaluated for various parameters.

### Material

(1) Feed

Normal diet: D12450B (Research Diets)

High-fat diet: D12451 (Research Diets)

(2) Ingested Sample

Preparation B: Doctor's Best Curcumin Phytosome with Meriva (a commercially available curcumin preparation)

Example 10: 0.1 mass % (in terms of curcumin) aqueous liquid of Preparation A (fine granules) (test group)
Non-treated group: normal diet and water
Control group: high-fat diet and water (by ad libitum drinking)
Preparation B group: high-fat diet and aqueous liquid of preparation B (by ad libitum drinking)
Example 10 group: high-fat diet and aqueous liquid of the preparation of Example 10 (by ad libitum drinking).

### Method for Analyzing Plasma Triglyceride Concentration

After the feeding period, the blood was sampled and centrifuged at 3000G at 4° C. for 15 minutes to obtain plasma.

The plasma was measured by a Fuji Dri-Chem 4000V biochemical automatic analyzer (Fujifilm, Tokyo, Japan).

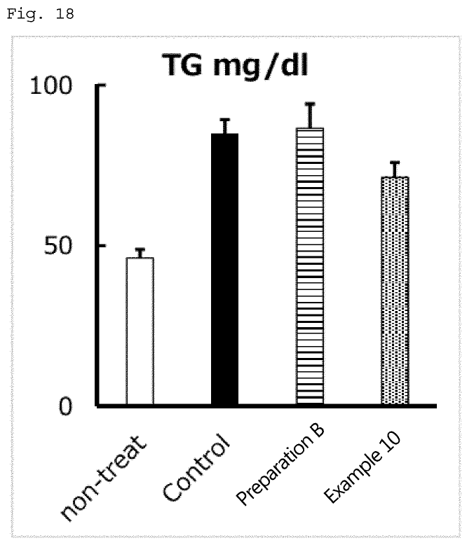

FIG. 18 shows the results.

### Test Example 16

### Measurement of Liver Weight and Measurement of ACOX1 Expression Level

### (1) Measurement of Liver Weight

After the mice subjected to the test of Example 10 were perfused with 5 ml of PBS or more to remove blood, liver weight was measured.

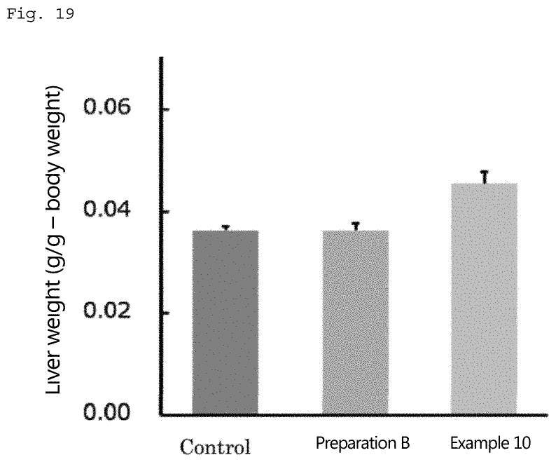

FIG. 19 shows the results.

### (2) Measurement of ACOX1 Expression Level

Using an RNeasy Plus Mini Kit (Qiagen, Calif., USA), mRNA was extracted from the liver obtained by dissection. The mRNA was reverse-transcribed into cDNA using a High-Capacity cDNA Reverse Transcription Kit (Thermo Fisher Scientific, Mass., USA).

Using the obtained cDNA as a template, a reaction liquid was prepared using a GeneAce SYBR qPCR Mix a Low ROX (Nippon Gene, Tokyo, Japan). The mRNA expression level of ACOX1 was determined by real-time PCR using a CFX384 (Bio-Rad Laboratories, CS, USA).

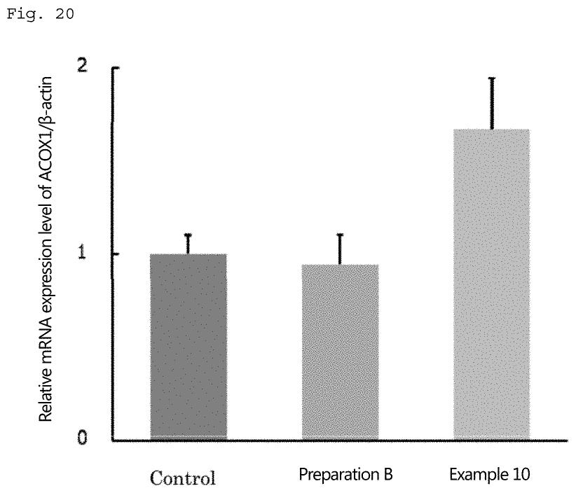

FIG. 20 shows the results.

The liver weight increase and the ACOX1 expression level increase shown in the results suggest that the curcumin preparation of the present invention promoted PPAR-α activation.

### Test Example 17

### Evaluation of Urinary Curcumin Level

The urinary curcumin level was evaluated by the following method.

### Test Method

Each curcumin preparation was orally, individually administered to rats at a dose of 100 mg/kg in terms of curcumin.

Urine was sampled over time, and the curcumin concentration in urine was analyzed.
Animal: 7-weeks-old male SD rats, 3 rats per group
Solution administered: 1% (in terms of curcumin) aqueous solution
Administration method: single oral administration (sonde method)

### Analytical Method

a) 10 μL of a 0.1 M acetate buffer (pH 5.0) was added to the sampled urine.

For the measurement of the concentration of curcumin, inclusive of glucuronide conjugate, 25 μL of β-glucuronidase was then added, and the resulting mixture was mixed by a 10 s vortex and enzymatically treated.

b) Subsequently, 50 μL of a 200 ng/mL emodin solution was added.
c) Subsequently, 500 μL of an ethyl acetate/methanol=95/5 solution was added, and the resulting mixture was vortexed for 1 minute.
d) Subsequently, the resulting mixture was centrifuged at 10000G at 4° C. for 5 minutes, and the supernatant was collected in a 2-mL tube.
The procedures c) and d) were further repeated twice, and the collected supernatants were combined in a 2-mL tube.

The combined supernatants were evaporated to dryness under nitrogen.

On the day of analysis, 200 μL of 80% methanol was added to the nitrogen-dried sample, and the resulting mixture was vortexed for 1 minute.

The resulting mixture was centrifuged at 10000G at 4° C. for 5 minutes, and the supernatant was collected.

The supernatant was filtered through a 0.45 μm membrane filter, and analyzed by UV detection (wavelength 420 nm).

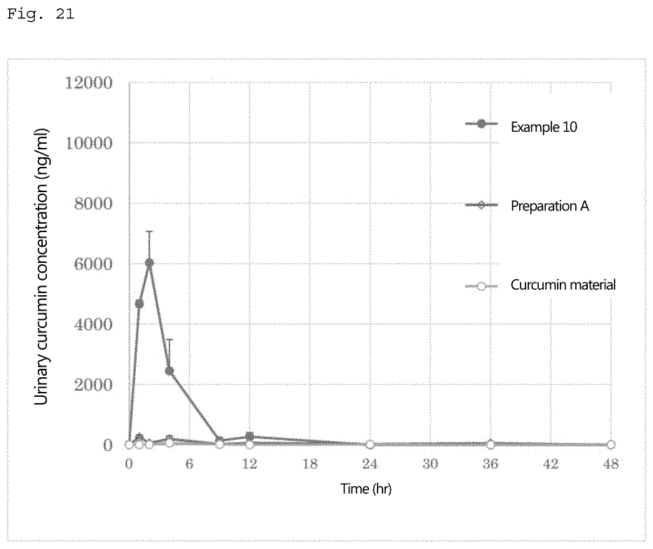

FIG. 21 shows the urinary curcumin concentration after administration of each curcumin preparation.

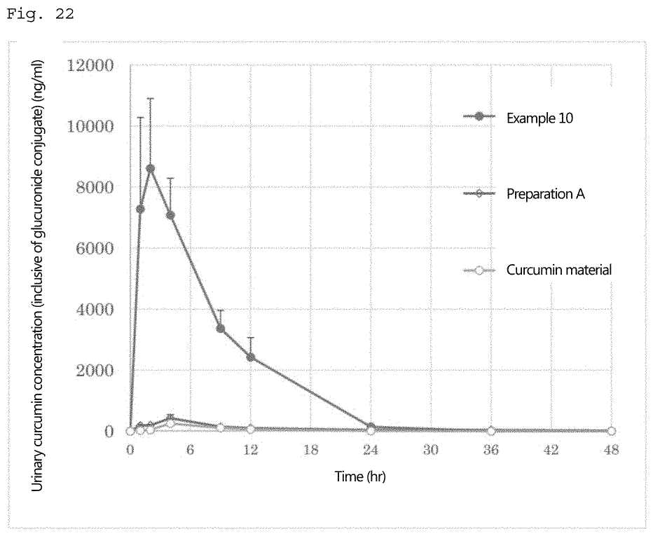

FIG. 22 shows the urinary curcumin (inclusive of glucuronide conjugate) concentration after administration of each curcumin preparation.

1. A method for treating or preventing a disease or symptom that benefits from curcumin absorption into cells, the method comprising administering a solid composition comprising:
(1) curcumin;
(2) hydrophilic polymer; and
(3) at least one nonionic surfactant selected from the group consisting of polyglycerol fatty acid esters, sucrose fatty acid esters, and lecithins,
to a subject in need thereof.

2. The method according to claim 1, wherein the disease or symptom is at least one member selected from the group consisting of:
(1) diseases or symptoms that benefit from modulation of one or more transcription factors selected from the group consisting of NF-κB, AP-1, STAT, Wnt/β-catenin, Notch-1, EGR-1, CREB-BP, WT-1, HIF, ERE, Nrf-2, PPAR-α, and PPAR-γ;
(2) diseases or symptoms that benefit from modulation of one or more cytokines selected from the group consisting of TNF-α, IL-2, IL-5, IL-6, IL-8, IL-12, IL-18, MCP-1, MIP-1α, and MaIP;
(3) diseases or symptoms that benefit from modulation of one or more receptors selected from the group consisting of IR, ER-α, H2R, HER-2, LDLR, ITR, FasR, EPCR, AR, EGFR, IL-8R, CXCR4, AHR, and DR-5;
(4) diseases or symptoms that benefit from modulation of one or more enzymes selected from the group consisting of Desaturase, GCL, AATF-1, ATFase, Telomerase, MMP, ATPase, GICL, COX-2, iNOS, NQO-1, 5-LOX, TMMP-3, DNA pol, Src-2, FPT, PhP D, GST, ODC, and ACOX-1;
(5) diseases or symptoms that benefit from modulation of one or more growth factors selected from the group consisting of HGF, CTGF, FGF, NGF, PDGF, TGF-β1, EGF, VEGF, and TF;
(6) diseases or symptoms that benefit from modulation of one or more kinases selected from the group consisting of FAK, AAPK, P60c-tk, EGFR-K, Ca²⁺PK, PTK, MAPK, IL-1R AK, PKB, PKA, PAK, JAK, ERK, PhK, and JNK;
(7) diseases or symptoms that benefit from modulation of one or more members selected from the group consisting of uPA, Bc1-2, Bc1-xL, VCAM-1, ICAM-1, ELAM-1, IAP-1, Hsp-70, Cyclin D1, MDRP, p53, and DEF-40;
(8) diseases or symptoms that benefit from inhibition of aggregation of amyloid β;
(9) diseases or symptoms that benefit from suppression of aggregation of α-synuclein;
(10) diseases or symptoms that benefit from a decrease of one or more members selected from the group consisting of ALT, AST, and γ-GTP;
(11) diseases or symptoms that benefit from inhibition of HAT activity of p300;
(12) diseases or symptoms that benefit from antioxidant effects; and
(13) diseases or symptoms that benefit from suppression of an increase in acetaldehyde concentration due to alcohol ingestion.

3. The method according to claim 1, wherein the treatment or prevention of a disease or symptom is at least one member selected from the group consisting of:
(1) treatment or prevention of cancers or tumors;
(2) treatment or prevention of diabetes;
(3) treatment or prevention of hyperglycemia;
(4) treatment or prevention of periodontitis;
(5) treatment or prevention of Alzheimer's disease or mild cognitive impairment;
(6) treatment or prevention of Parkinson's disease;
(7) treatment or prevention of nerve disorder;
(8) treatment or prevention of inflammation;
(9) treatment or prevention of amyloidosis;
(10) protection of liver function;
(11) treatment or prevention of heart failure;
(12) treatment or prevention of myocardial infarction;
(13) treatment or prevention of muscle fatigue;
(14) protection of kidney function;
(15) treatment or prevention of osteoporosis;
(16) treatment or prevention of depression;
(17) treatment or prevention of multiple sclerosis;
(18) treatment or prevention of ischemia; and
(19) treatment or prevention of hangover symptoms after alcohol ingestion.

4. The method according to claim 1, wherein the treatment or prevention of a disease or symptom is at least one member selected from the group consisting of suppression of cholesterol elevation, suppression of triglyceride elevation, suppression of chylomicron elevation, suppression of blood pressure elevation, suppression of blood glucose elevation, anti-allergy, and suppression of body fat.

5. The method according to claim 1, wherein the hydrophilic polymer is at least one member selected from the group consisting of polyvinylpyrrolidone, hydroxypropyl cellulose, and hydroxypropyl methylcellulose.

6. The method according to claim 1, wherein the nonionic surfactant is a polyglycerol fatty acid ester.

7. The method according to claim 1, wherein the administration is oral administration, administration through gastrointestinal tract, transdermal administration, or pulmonary administration.

8. The method according to claim 1, wherein a pharmaceutical product, quasi-drug, health food, food with function claims, dietary supplement, food with nutrient function claims, nutritional supplement, food for special dietary use, or a food for specified health use comprising the solid composition is administered in the method.

9. The method according to claim 1, wherein the solid composition is substantially free from crystalline curcumin.

10. The method according to claim 1, wherein the solid composition comprises the curcumin in an amount of 1 to 60 mass %.

11. The method according to claim 1, wherein the solid composition comprises the curcumin in an amount of 10 to 35 mass %.

12. The method according to claim 1, wherein the hydrophilic polymer is solid at room temperature.

13. The method according to claim 1, wherein the hydrophilic polymer has a glass transition temperature of about 50° C. to about 180° C.

14. The method according to claim 1, wherein the solid composition comprises the hydrophilic polymer in an amount of 5 to 90 mass %.

15. The method according to claim 1, wherein the solid composition comprises the hydrophilic polymer in an amount of 40 to 90 mass %.

16. The method according to claim 1, wherein the solid composition comprises the nonionic surfactant an amount of 5 to 90 mass %.

17. The method according to claim 1, wherein the solid composition comprises the nonionic surfactant an amount of 10 to 40 mass %.
